(19) United States
(12) Patent Application Publication
(10) Pub. No.: US 2018/0110570 A1
(43) Pub. Date: Apr. 26, 2018

(54) Static and Dynamic Visual Depictions of Behavior of Participants in Healthcare Markets

(71) Applicant: Allevion, Inc., Aventura, FL (US)

(72) Inventors: Arnon Krongrad, North Miami, FL (US); Kimberly Susan Langer, Hallandale Beach, FL (US)

(21) Appl. No.: 15/782,524

(22) Filed: Oct. 12, 2017

Related U.S. Application Data
(60) Provisional application No. 62/411,749, filed on Oct. 24, 2016.

(51) Int. Cl. A61B 34/20; G06F 19/00; G06Q 30/06; G06F 17/30; A61B 90/00

(52) U.S. Cl. CPC A61B 34/20; G06F 19/3443; G06Q 30/0629; G06F 3/0482; A61B 90/39; A61B 2090/3937; G06F 17/30241

(57) ABSTRACT

An example method for visually depicting geographic distribution of facilities or participants in a market for invasive or operative procedures includes: displaying a map of a geographic area; defining a plurality of markers on the map, each marker being associated with an invasive or operative procedure, a facility, a patient, a healthcare provider, equipment, or other participant; displaying each marker associated with a bundled invasive or operative procedure at a geographical location of a facility at which the procedure is to be performed and displaying each marker associated with a patient, healthcare provider, equipment, or other participant at a geographical location at which the patient, healthcare provider, equipment or other participant originates; calculating one or more distances between the patient, healthcare provider, equipment, or other participant and the facility; determining an activity density of procedures performed; and presenting static or dynamic visual depictions on the map representing participant behavior.

### RELATED APPLICATION(S)

This application is related to U.S. patent application Ser. No. 14/657,146 filed on Mar. 13, 2015, the entirety of which is hereby incorporated by reference.

### BACKGROUND

The high cost and limited availability of invasive and/or operative procedures may present barriers to their delivery to patients, be it directly or indirectly through third-party payors. High price and limited availability cause patients to delay and altogether skip the care they need.

Delivery of an invasive and/or operative procedure requires the carefully synchronized, coordinated, matched, and complete bundling of highly-specialized and specific human and material components, including providers, such as surgeons, anesthesiologists, and pathologists; environments, such as operating rooms and endoscopy suites; and equipment, such as power drills; and instruments. These components of invasive and/or operative procedures may be found in uncoordinated and different locations. This leads to inefficiencies and price increases.

Facilities for performing invasive and/or operative procedures are often geographically fixed: they cannot easily be moved. Participants in the surgical event are geographically free. The ways in which participants in the surgical event move and the reasons for their movement are poorly understood.

Markets for invasive and/or operative procedures allow deeper insights regarding the ways in which participants move and the reasons for their movement. Such insights can enable optimization of the design and distribution of invasive and/or operative procedures in ways that help to simplify access to care.

### SUMMARY

Embodiments of the disclosure are directed to an example method for visually depicting the location and/or points of origin of facilities and/or participants in a market for invasive and/or operative procedures includes: displaying a map of a geographic area; defining a plurality of markers on the map, each marker being associated with an invasive and/or operative procedure, a related facility, a patient, a healthcare provider, equipment, and/or other participant; displaying each marker associated with a bundled invasive and/or operative procedure at a geographical location of a facility at which the procedure is to be performed and displaying each marker associated with a patient, healthcare provider, equipment, and/or other participant at a geographical location at which the patient, healthcare provider, equipment and/or other participant originates; calculating one or more distances between the patient, healthcare provider, equipment, and/or other participant and the facility; determining an activity density of procedures performed; and manually and/or automatically presenting static and/or dynamic visual depictions on the map representing participant behavior.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 shows an example system that supports bundling medical provider services.

FIG. 2 shows example modules of the web server computer of FIG. 1.

FIG. 3 shows an example user interface that may be rendered on a client computer of FIG. 1.

FIG. 4 shows an enlarged view of the interactive map of the user interface of FIG. 3.

FIG. 5 shows enlarged views of the tables of the user interface of FIG. 3.

FIG. 6 shows another example user interface that may be rendered on a client computer of FIG. 1.

FIG. 7 shows an enlarged view of the interactive map of the user interface of FIG. 6.

FIG. 8 shows enlarged views of the tables of the user interface of FIG. 6.

FIG. 9 shows yet another example user interface that may be rendered on a client computer of FIG. 1.

FIG. 10 shows an example certificate of complications protection.

FIG. 11 shows another example certificate of complications protection.

FIG. 12 shows an example user interface for financing options.

FIG. 13 shows a flowchart for an example method implemented on the web server computer of FIG. 1 for bundling invasive and/or operative procedures.

FIG. 14 shows a flowchart for an example method for providing credentials and privileges for a provider.

FIG. 15 shows example physical components of the web server computer of FIG. 1.

FIG. 16 shows an alternative example user interface that may be rendered on the client computer of FIG. 1.

FIG. 17 shows an enlarged view of the interactive map of the user interface of FIG. 16.

FIG. 18 shows alternative example modules of the web server computer of FIG. 1.

FIG. 19 shows an example user interface for displaying a geographic distribution of facilities and participants in a bundled invasive and/or operative procedure market.

FIG. 20 shows an alternate view of the user interface of FIG. 19.

FIG. 21 shows an alternate view of the user interface of FIG. 19.

FIG. 22 shows another example dynamic user interface for displaying an activity density in a bundled invasive and/or operative procedure market.

FIG. 23 shows another view of the example dynamic user interface of FIG. 22.

FIG. 24 shows a flowchart for an example method of presenting a geographic distribution of facilities and/or participants in a market for invasive and/or operative procedure bundles.

### DETAILED DESCRIPTION

The present disclosure is directed to systems and methods for bundling medical provider services, facilities, implants and equipment in order to produce and present a plurality of new choices of service bundles and increase convenience and transparency, and reduce price to the individual or entity at financial risk for the surgery. Facing the bundled service choices produced by these systems and methods, an end user who is looking for an invasive and/or operative procedure may select from a plurality of bundled service choices the one that best meets his/her needs. Components of bundles of invasive and/or operative procedures can include: an appropriately credentialed and privileged physician to perform the procedure, a facility in which the procedure may be performed, a date for the procedure, and a price for the procedure.

Some procedures that do not create a break in the skin, such as colonoscopies, are commonly referred to as “invasive.” Other procedures, for example surgical procedures that create a break in the skin, are commonly referred to as “operative.” The procedures described herein include both invasive and/or operative types such as knee replacements, hip replacements, hysterectomies, sleeve gastrectomy, cardiac catheterization, and colonoscopies; other procedures are possible.

The providers that perform the procedures may include surgeons, surgical assistants, physician assistants, anesthesiologists, nurse anesthetists, gastroenterologists, pathologists, radiology technicians, etc. Such providers are credentialed and privileged at the facility at which the procedure is to be performed; primary documents, such as medical licenses, board certifications, etc. are verified.

The facilities at which invasive and/or operative procedures may be performed include hospitals, ambulatory surgery centers, endoscopy centers, radiology centers, medical centers and physician offices. The facilities are generally located in a well-defined geographical area, such as a state. However, the facilities may be in various locations within the geographical area. Equipment used for the procedure may be resident at the facility or may be transported to the facility from other locations for the operative episode.

The bundled services are presented to the end user via a computer interface, typically via a web page or data import into a client legacy environment that may be accessed by the end user. As discussed in more detail later herein, the web page provides a map showing the specific location of the bundled service, provides detailed information regarding the bundles and permits a selection of a bundle by the potential patient.

The systems and methods also provide for protection against economic losses due to complications that may arise during and/or after the invasive and/or operative procedure, to address the psychological barrier to care—fear of complications—and to give the patient peace of mind (“complications protection”). In this example, protection against complication-related financial losses is automatically included in those procedures for which it is available. In other examples, the protection is provided as an option. In addition, the systems and methods also provide access to financing options for the potential patient to minimize financial barriers to care.

The systems and methods are directed to three types of clients: uninsured patients, insured patients, self-insured employers (i.e., Carnival Cruise Lines) and other third party payors. One example of a third party payor is an insurer for the patients. An example of an insurer is Blue Cross Blue Shield of Florida, also known as Florida Blue.

The systems and methods make use of a computer algorithm to compile information regarding available providers, facilities, implants and equipment and to create bundled services of invasive and/or operative procedures that are made available to the end user via the web site or data import. The web site is searchable, for example permitting an uninsured end user to search for availability for a specific procedure at a specific location at a specific price and time. The computer algorithm provides for sorting and filtering of various combinations of providers, locations and price.

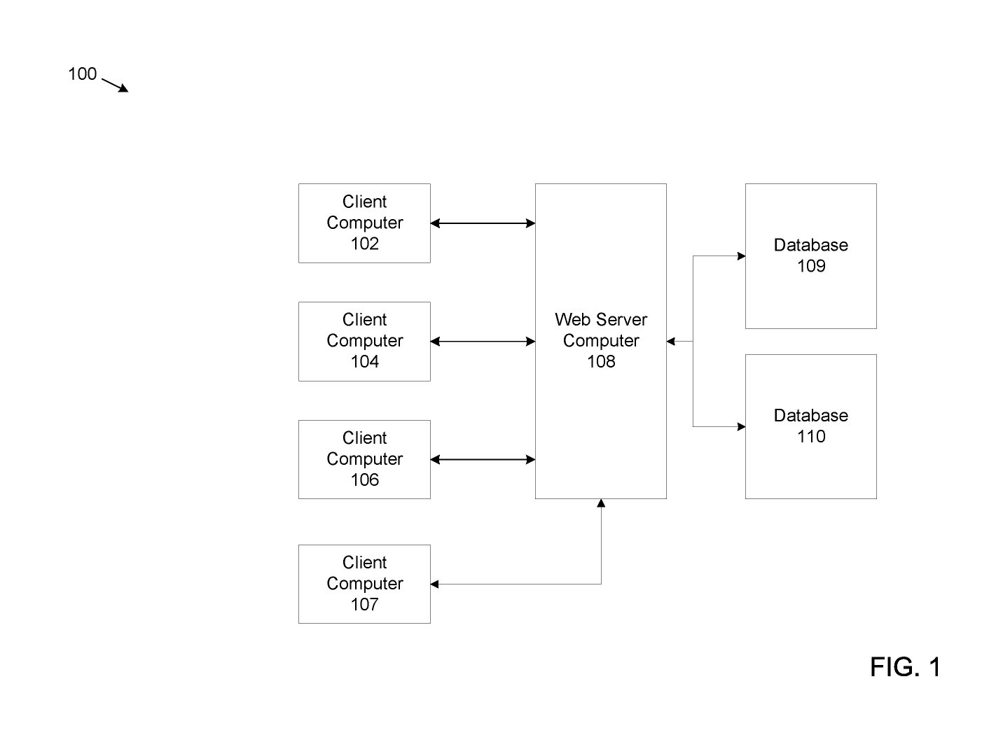

FIG. 1 shows an example system 100 that supports bundling of invasive and/or operative procedure bundles. System 100 includes client computers 102, 104, 106, 107, web server computer 108 and database 110.

Client computers 102, 104, 106 and 107 may be desktop, laptop or tablet computers, smart telephones, and/or other interactive digital products. Client computers 102, 104, 106 and 107 permit a user to access a web site that includes information on the invasive and/or operative procedure bundles. For the example system 100, client computer 102 is used by an uninsured patient, client computer 104 is used by an insured patient, client computer 106 is used by a payor, for example by an employee of Florida Blue, and client computer 107 is used by a member of a third party payor plan via the payor's user portal (as described below). More or fewer client computers may be used.

Web server computer 108 is a server computer that hosts the web site that may be accessed via client computers 102, 104 and 106 and 107. Database 109 is a central repository used to store patient information submitted via the online consultation request form. The web site provides information regarding the invasive and/or operative procedure bundles that may be rendered on a web browser of client computers 102, 104, 106 and 107. Database 110 also includes one or more computer algorithms that are used to compile the invasive and/or operative procedure bundles from information that may be pushed to and stored on web server computer 108, on database 110 or on other server computers or databases.

Database 110 is a database that includes information on providers, facilities, vendors and equipment, as well as pricing for the same. Providers include available surgeons, assistants, anesthesiologists and other physicians. Facilities include available hospitals, medical centers and physician offices. Implants include any necessary components needed to perform a specific invasive and/or operative procedure. Equipment includes any equipment needed to perform a specific invasive and/or operative procedure. Some or all of the equipment may be located at one of the facilities. Some or all of the equipment may be located elsewhere and may need to be transported to one of the facilities. Algorithms referenced in 0034 are used to identify and fulfill equipment needs for a specific invasive and/or operative procedure at a specific facility.

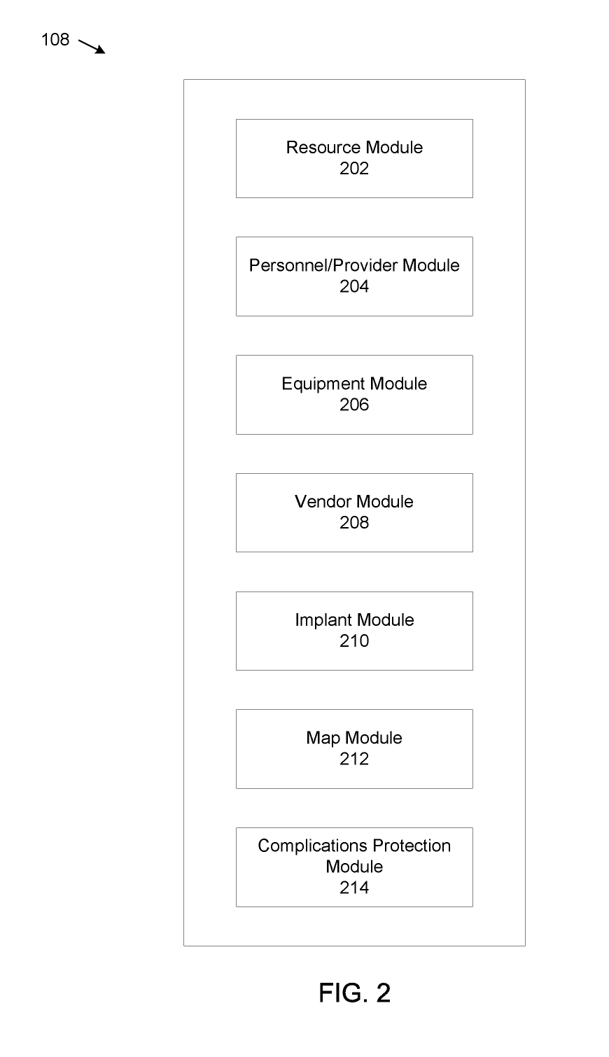

FIG. 2 shows example module of web server computer 108. The example modules include a resource module 202, a personnel module 204, an equipment module 206, a vendor module 208, an implant module 210, a map module 212 and a complications protection module 214.

Other modules are possible. For example, another module can be programmed to track dates and provide pricing for different invasive and/or operative procedure bundles based upon the particular date and time. In yet other examples, such a module can assist in scheduling of the different aspects of the invasive and/or operative procedure bundles, including patient visits and/or location/surgical components.

The example resource module 202 is programmed to identify medical facilities including qualified, credentialed, and privileged providers, operating room capabilities, associated procedure fees and facility assets that may be used as part of the invasive and/or operative procedure bundles. The medical facilities and associated characteristics thereof are obtained from database 110.

The example personnel module 204 is programmed to identify medical providers that may be used as part of the invasive and/or operative procedure bundles. As part of the identification process, the personnel module 204 also verifies credentials for the identified medical providers, determines facilities where the medical providers have privileges and determines availability for the medical providers. Personnel may perform more than one operative or invasive procedure at more than one facility. Personnel module 204 also includes the fee for the specific provider at a specific location for a specific procedure. Personnel module 204 also includes information such as personal statements and administrative contacts. More elements may be added.

In some examples, the personnel module 204 also captures other metrics about the medical providers. For example, in the context of a physician, the personnel module 204 can be programmed to capture outcome-related quality metrics over time on a per-location or per-physician basis. One example of such a metric is a clinical outcome like intraoperative blood loss. Other objective and/or subjective metrics can be tracked and provided to the end user when selecting among bundles.

The example equipment module 206 is programmed to identify equipment that is needed to perform the invasive and/or operative procedures. The equipment module 206 also determines location and availability of the identified medical equipment, specifically determining whether the identified equipment is located and available at the facilities identified by the resource module 202.

The example vendor module 208 is programmed to identify equipment and implant vendors that may be used to obtain medical equipment and implantable devices that may not be available at the facilities identified by the resource module 202. The identified equipment vendors are also vendors that have the capability of transporting the medical equipment to facilities where the medical equipment is needed.

The example implant module 210 is programmed to identify availability of implants that may be needed during the invasive and/or operative procedures. Examples of implants that may be needed include artificial knee replacements and artificial hip replacements. Other implants may be used. It is also noted that surgeons have preferences regarding the brand/type of implant. This characteristic is factored into creation of the bundle.

In this embodiment, the resource module 202, personnel module 204, equipment module 206, implant module 210 and vendor module 208 are used to create the price for the bundle of medical services. In this example, the price is the actual cost to the patient for the medical service (including any fees associated with the use of the system 100. For example, each facility may charge a different amount for use of a particular asset, such as an operating room. Further, each medical provider can charge different amounts based upon, for example, the type of procedure and location at which the procedure will occur.

Further, in this embodiment, the credentialing and privileging of the medical provider at each of the facility locations is tracked. In one example, there is an automated process that allows the provider to obtain privileges at multiple facilities. This allows for greater flexibility and efficiency in the matching of the locations at which a provider can deliver services resulting in new services previously unavailable to the end user.

The example map module 212 is programmed to render an interactive map on a user interface of client computers 102, 104, 106 and 107. The interactive map shows locations where invasive and/or operative procedure bundles are available and provides related details.

The example complications protection module 214 is programmed to compile complications protection that may be presented to patients. The complications protection provides varying levels of coverage to provide patients with protection from the financial losses associated with complications arising during and after the procedures.

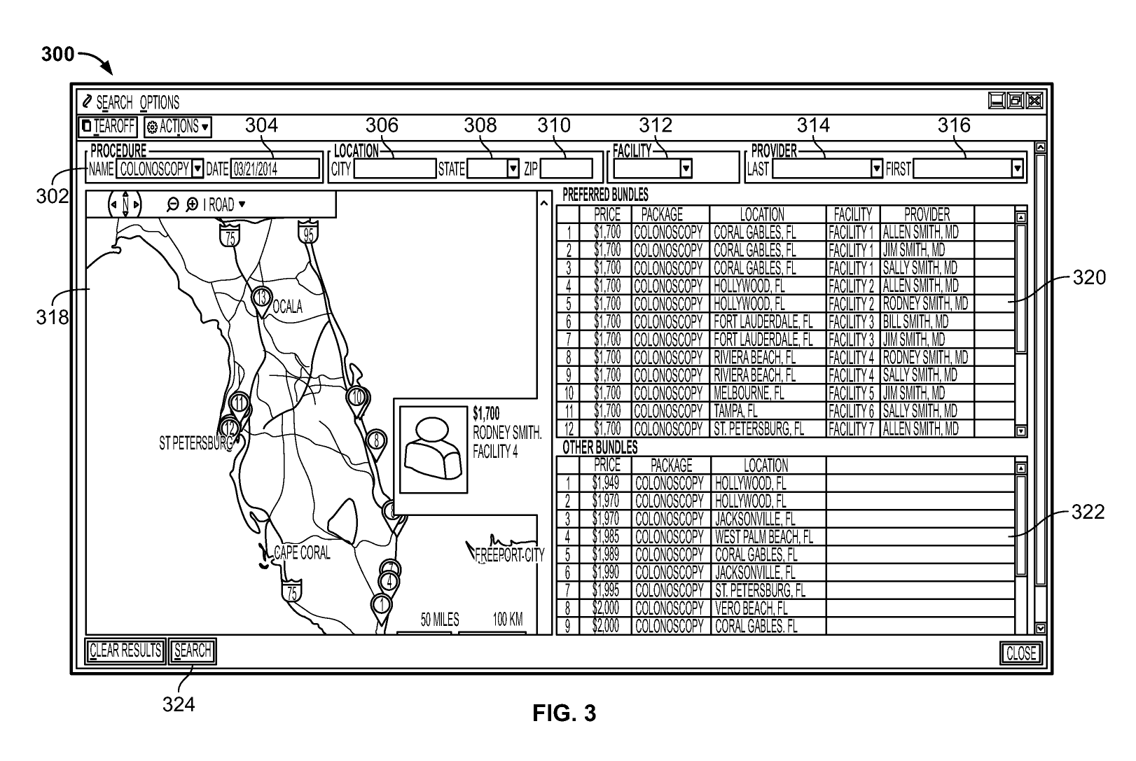

FIG. 3 shows an example user interface 300 that may be rendered for the end user on a web browser of client computers 106. The end user may be an uninsured patient or a third party payor, such as an insurance company. The example user interface 300 shows a result of a search for particular procedure bundles (e.g., a colonoscopy) in a specific geography (e.g., the state of Florida).

The user interface 300 includes search fields for the service bundle including search fields for a procedure 302, a date 304, a location including a city 306, a state 308 and a zip code 310, a facility 312 and a provider including a last name 314 and first name 316. The user interface 300 also includes an interactive map 318, a table 320 of service bundles from preferred bundles, a table 322 of service bundles from other bundles, and a search button 324.

The user interface 300 shows a result of search for bundled invasive and/or operative procedures on a specified date (e.g., Mar. 21, 2014. The end user at client computer 102 or 106 selects the procedure from procedure field 302. A drop down list box (not shown in FIG. 3) permits the end user to select the procedure from a list of available procedures. The date field 304 permits the payor to enter a specific date for the procedure. The payor then selects search button 324. The payor can also search for and then possibly filter and sort a specific location, facility and provider.

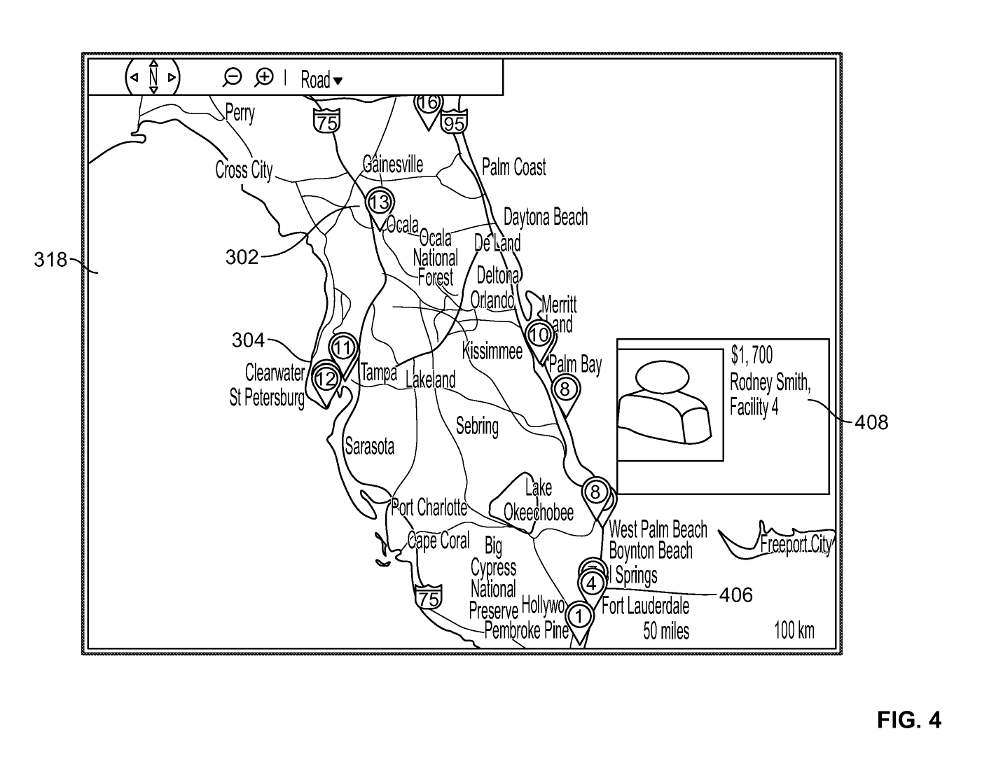

FIG. 4 shows an enlarged view of interactive map 318. Interactive map 318 is displayed as result of a search for available procedure bundles for any given date in the state of Florida. As shown in FIG. 4, each available bundle is indicated by a numbered marker. For example, marker 402 shows a marker numbered 13, indicated that a colonoscopy bundle is available on Mar. 21, 2014 in Ocala, Fla. Similarly, marker 404 shows a marker numbered 12, indicating an available colonoscopy bundle in St. Petersburg, Fla. and marker 406 shows a marker numbered 4, indicating an available colonoscopy bundle in Riviera Beach, Fla. In these examples, the example marker numbers 13, 12 and 4 correspond to identifiers for specific medical facilities.

When any one of these markers is selected, a pop-up window is displayed providing more information about the bundle at the marker location. For example, when marker 406 is selected, an example pop-up window 408 is displayed. Pop-up window 408 shows that a colonoscopy bundle at Riviera Beach Fla. is to be performed at facility 4 by Dr. Rodney Smith. The price of the procedure, including complications protection is $1,700. Other information, such as details about the service provider (e.g., details about the surgeon and facility) may also be provided.

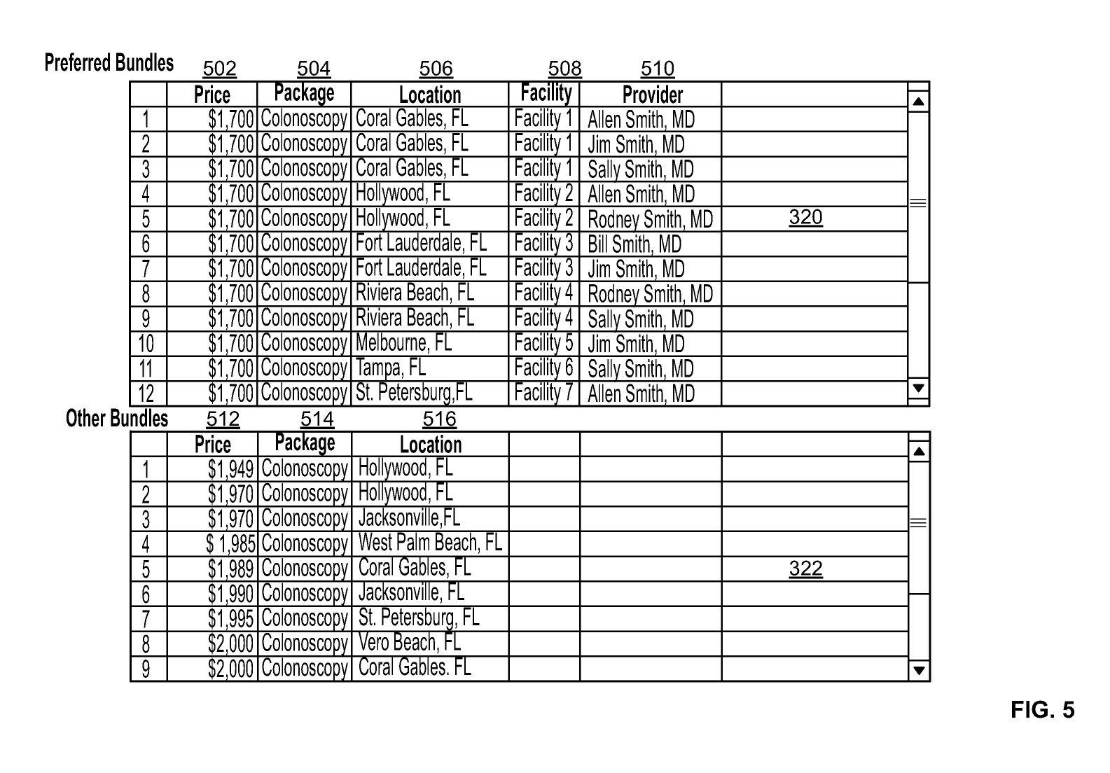

FIG. 5 shows enlarged views of example tables 320 and 322. Table 320 shows price comparisons for different service bundles for a preferred provider. Each bundle consists of a price (502) for a specific procedure (504), in this case a colonoscopy, at a specific location (506), at a specific facility (508) with a specific provider (510). The provider is a physician performing the procedure. Table 320 includes 12 procedure bundles. As shown, bundles are available at seven different cities within the state of Florida and at seven different facilities, each facility corresponding to a specific city.

In some examples, the tables 320, 322 are presented to end users or other users of the system (see “white-label” implementation described below) below. In other examples, the tables 320, 322 are not presented to end users in the format shown.

Each service bundle within a specific city includes a different provider. However, certain providers may perform procedures at multiple locations. For example, service bundles are provided for Allen Smith at facility 1 in Coral Gables, at facility 2 in Hollywood and at facility 7 at St. Petersburg.

Table 322 provides price comparisons for the same colonoscopy procedure bundle but for other providers. Table 322 includes nine different options including the price (512), specific procedure (514) and the location (516). As shown in table 322, prices for the other provider bundles range from $1,949 to $2,000, each of which is higher than the $1,700 bundle price.

In one example, the tables 320, 322 represent the bundles of medical services associated with different health plans. In other examples, the tables can be used to present prices associated with bundles that fall outside of medical coverage of a patient, along with bundles associated with the patient's medical insurance coverage.

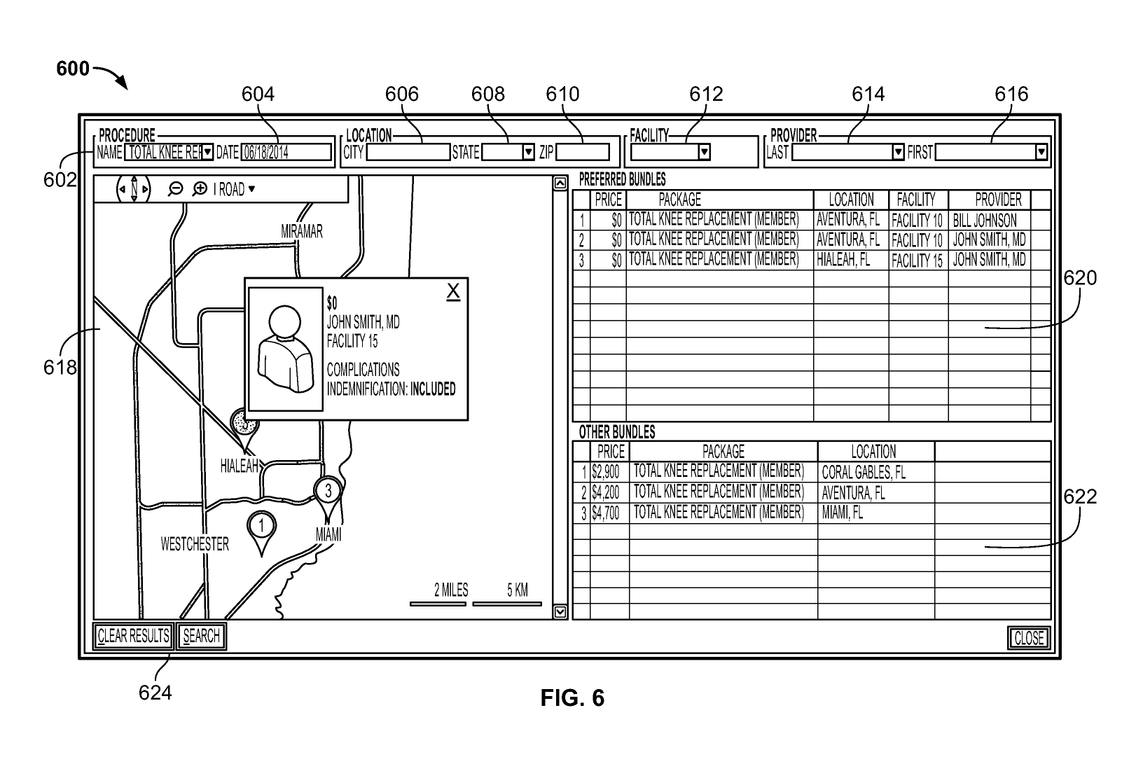

FIG. 6 shows an example user interface 600 for an insured patient that may be rendered on a web browser of client computer 104. The example user interface 600 shows a result of a search for a total knee replacement bundle in the state of Florida with out of pocket amounts for an insured patient rendered using electronic data interchange (EDI) benefit determination transactions.

With user interface 600, a search can be made for a specific procedure (602), a specific date (604) a specific location including a city (606), a state (608) and a zip code (610, a specific facility (612) and a specific provider including a last name (614) and a first name (616) and other provider-specific details. User interface 600 also includes an interactive map 618, a table 620 of service bundles from preferred bundles, a table 622 of service bundles from other bundles and a search button 624.

The user interface 600 shows a result of search for bundled total knee replacement on Mar. 21, 2014. The insured patient at client computer 104 selects the total knee replacement procedure from procedure field 602. A drop down list box (not shown in FIG. 6) permits the insured user to select the total knee replacement procedure from a list of available procedures. The date field 604 permits the insured user to enter a specific date for the procedure. Bundle pricing may fluctuate depending upon the date of the query. Price date sensitivity is accommodated in the bundle generation. The insured user then selects search button 324. The insured user can also search for a specific location, facility and provider.

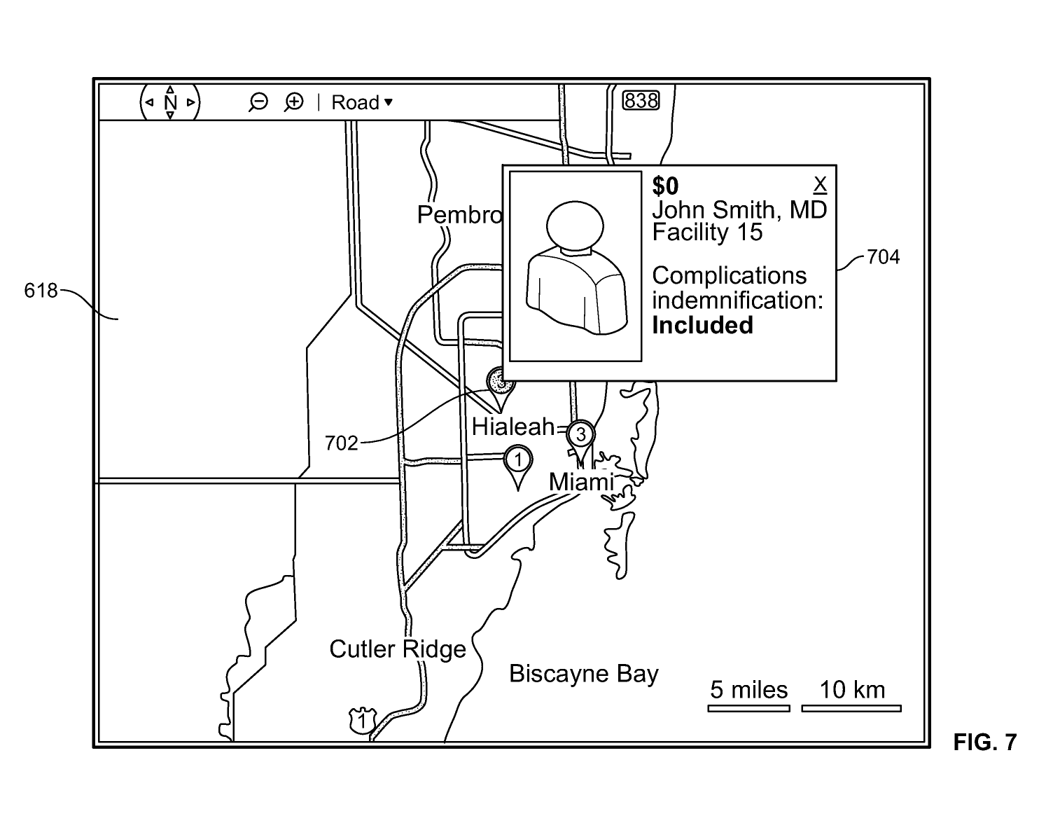

FIG. 7 shows an enlarged view of interactive map 618. Interactive map 618 is displayed as result of a search for available total knee procedure bundles on Mar. 21, 2014 in the state of Florida. As shown in FIG. 7, each available bundle is indicated by a numbered marker. For example, marker 702 shows a marker numbered 3, indicated that a total knee replacement bundle is available on Mar. 21, 2014 in Hialeah, Fla.

When marker 702 is selected, a pop-up window is displayed providing more information about the bundle at the marker 702 location. For example, when marker 702 is selected, an example pop-up window 704 is displayed. Pop-up window 704 shows that a total knee replacement procedure bundle at Hialeah Fla. is to be performed at facility A by Dr. John Smith. The pop-up window 704 also shows that the price of the procedure includes complications protection.

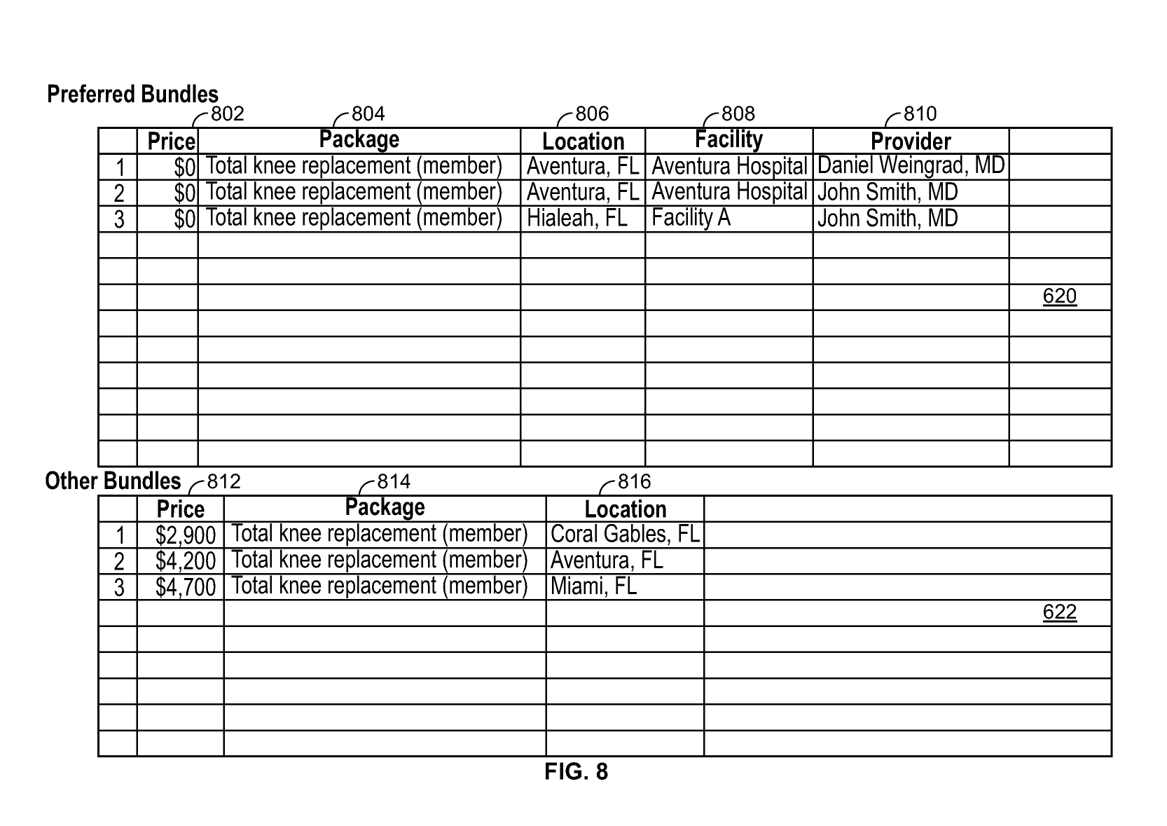

FIG. 8 shows enlarged views of example tables 620 and 622. Table 620 shows price comparisons for different service bundles for a preferred provider. Each bundle consists of a price (802) for a specific procedure (804), in this case a total knee replacement, at a specific location (806), at a specific facility (808) with a specific provider (810). The provider is a physician performing the procedure.

Table 620 includes three bundles. Because table 620 is for an insured patient, the price 802 represents an out-of-pocket cost for the patient based upon that patient's insurance benefits. For example, the patient may have no out-of-pocket costs, such as a deductible or cost share, for a particular invasive and/or operative procedure. As shown in FIG. 8, under this kind of benefit design, for preferred bundles the associated out-of-pocket cost for each is zero, whereas for other bundles the associated out-of-pocket cost for each is other than zero. These out-of-pocket costs are automatically calculated based upon patient-specific financial data kept by and procured from the insurance company using standardized Electronic Data Interchange (EDI) transactions and provided in the patient-specific tables 620, 622.

As shown, service bundles are provided at two different cities within the state of Florida and at two different facilities, each facility corresponding to a specific city. Each service bundle within a specific city includes a different provider. However, certain providers may perform procedures at multiple locations. For example, service bundles are provided for Dr. John Smith at Aventura Hospital in Aventura, Fla. and at Facility A at Hialeah, Fla.

Table 622 provides price comparisons for total knee replacement service bundles. Table 622 includes three different options including the price (812), specific procedure (814) and the location (816). As shown in table 622, prices for the other bundles range from $2,900 to $4,700.

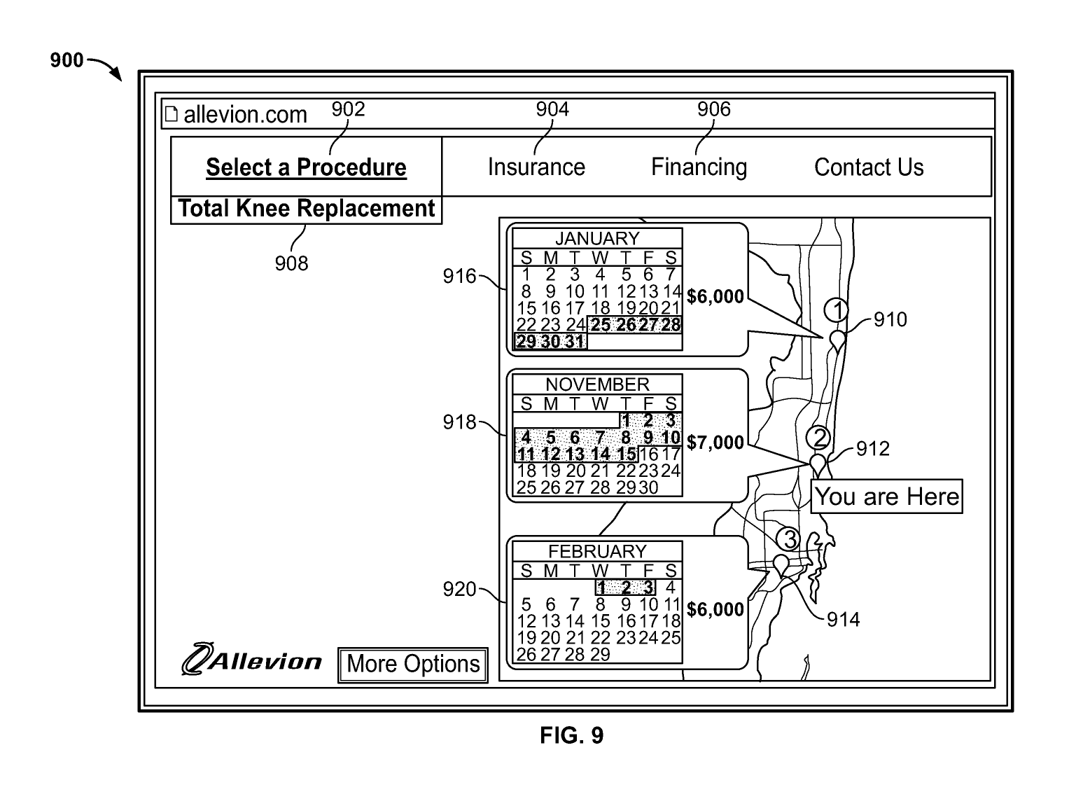

FIG. 9 shows an example alternative user interface 900 for an uninsured patient that may be rendered on a web browser of client computer 102. The alternative user interface 900 may be used in some embodiments. The example user interface 900 shows a result of a search for total knee replacement bundles in the state of Florida.

The example user interface 900 includes link 902 for selecting a procedure, link 904 for selecting complications protection and a link 906 for selecting financing options. When procedure 908 for a total knee replacement is selected, the user interface display shown in FIG. 8 is rendered on the web browser of client computer 102.

As shown in FIG. 9, an interactive map is displayed showing options for the uninsured patient. The interactive map displays markers 910, 912 and 914. Each marker corresponds to a city in which the total knee replacement bundle is available and the price with which it is associated. For example, marker 910 indicates Boynton Beach, Fla., marker 912 indicates Fort Lauderdale, Fla. and marker 914 indicates Coral Gables, Fl.

In the embodiment shown in FIG. 9, an information area is displayed for each of markers 910, 912 and 914. Information area 916 is associated with marker 910, information area 918 is associated with marker 912 and information area 920 is associated with marker 914. Each information area includes a calendar showing highlighted days on which the procedure may be performed and a price for the procedure. The uninsured patient can click a date on one of the information areas and sign up for the bundle on the date and location selected.

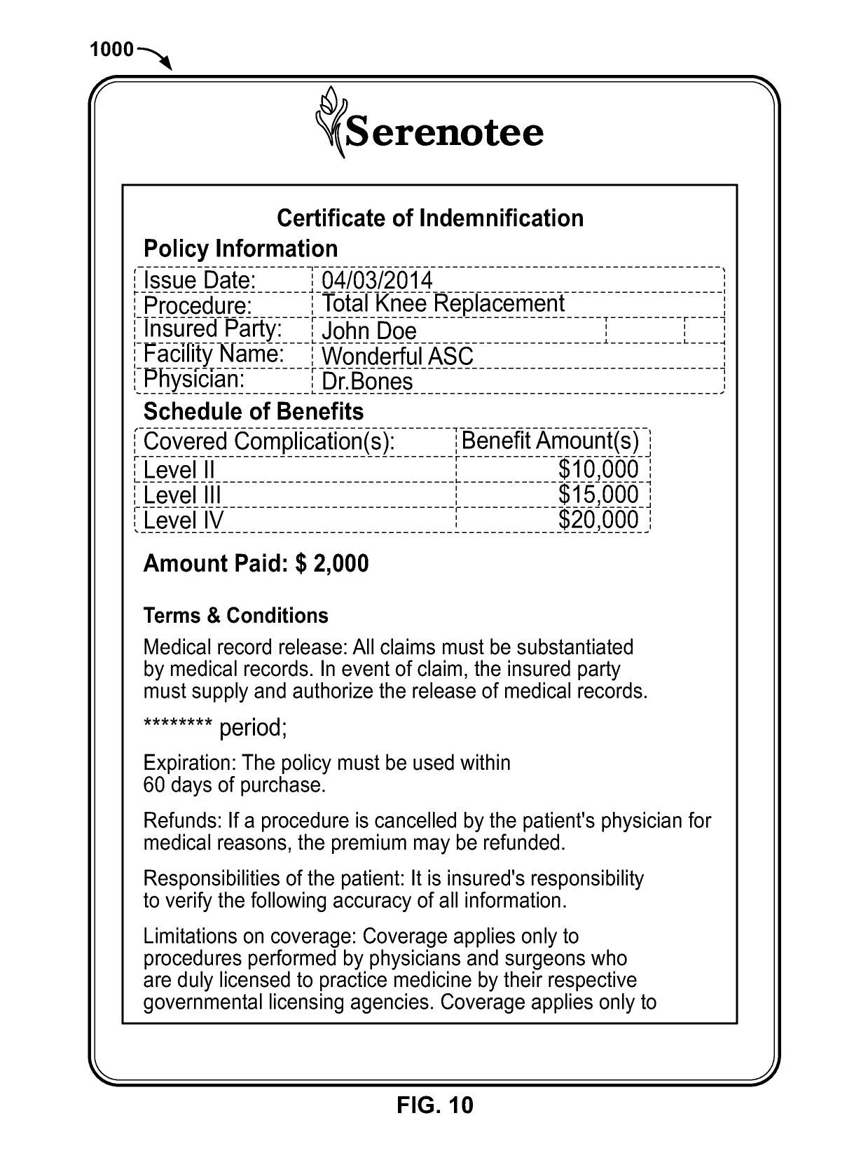

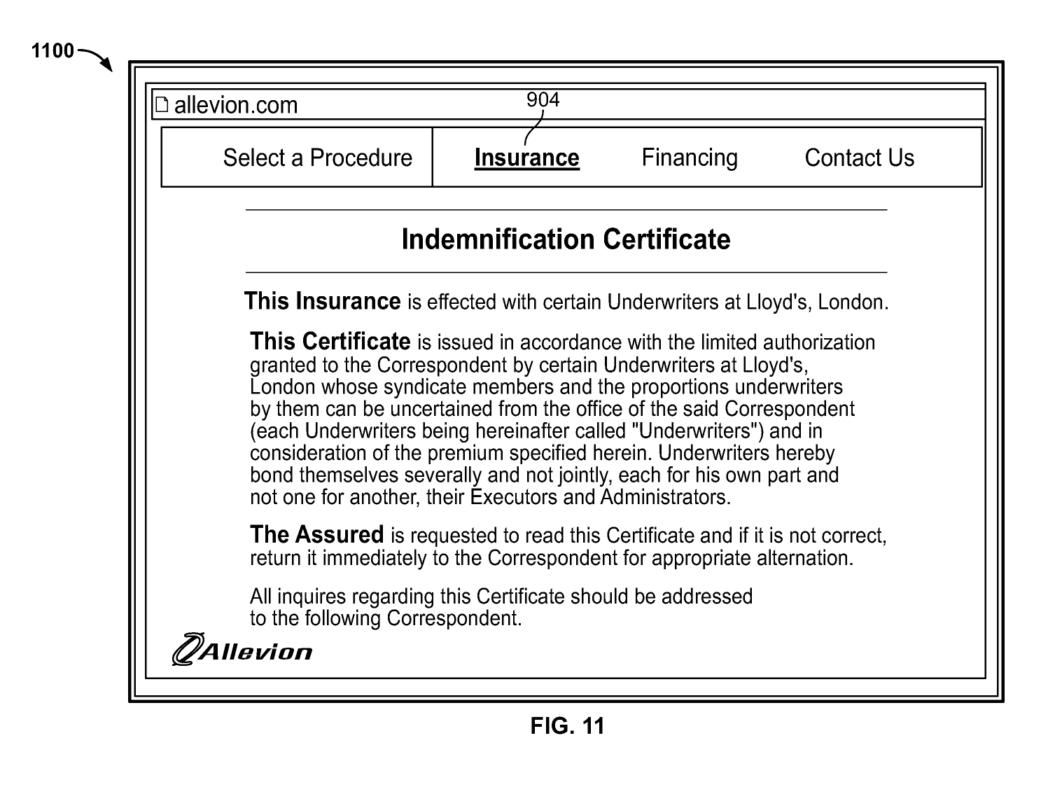

User interface 900 also permits the uninsured user to sign up for complications protection. The complications protection provides financial protection against complications associated with the procedure. FIG. 10 shows an example certificate of complications protection 1000. The example certificate of complications protection 1100 shows that the user paid $2,000 for the coverage. Also shown are policy information, a schedule of benefits and terms and conditions of the coverage. An example certificate of complications protection is shown in FIG. 11.

In examples provided herein, the prices of complications protection are developed using a pre-operative assessments of risks of pre-operative and intra-operative accidents and/or complications adversely affecting the health and/or functional status of patients going for operative and/or invasive procedures to pre-operatively price and qualify patients for insurance against possible accidents and/or complications.

In one example, this assessment involves two steps. The first step, risk assessment, identifies the types and severities of accidents and complications that are possible for a given operative or invasive procedure and produces an estimation of their likelihood. The risk assessment is determined upon analysis of relevant human and physical factors relating to the patient, medical personnel, e.g. surgeon and anesthesiologist, type of procedure, facility, and more.

The second step, pricing and qualification for insurance against accidents and/or complications of operative and/or invasive procedures, can apply to the output of the first step and other similar real-world scenarios and assessments, wherein a financial model and system is produced. The financial model, which can rely on the first step, forms risk pooling from individual patients about to have operative and/or invasive procedures. The newly formed risk pool and associated individual policy premiums provide the funds for payments to patients who have suffered accidents and/or complications and have made claims on that basis.

In the example shown, the complications protection is provided according to a set premium price that is added to the total price of the clinical service bundle that is presented to the patient. In other examples, the complications protection is provided as an option for the patient to select as desired.

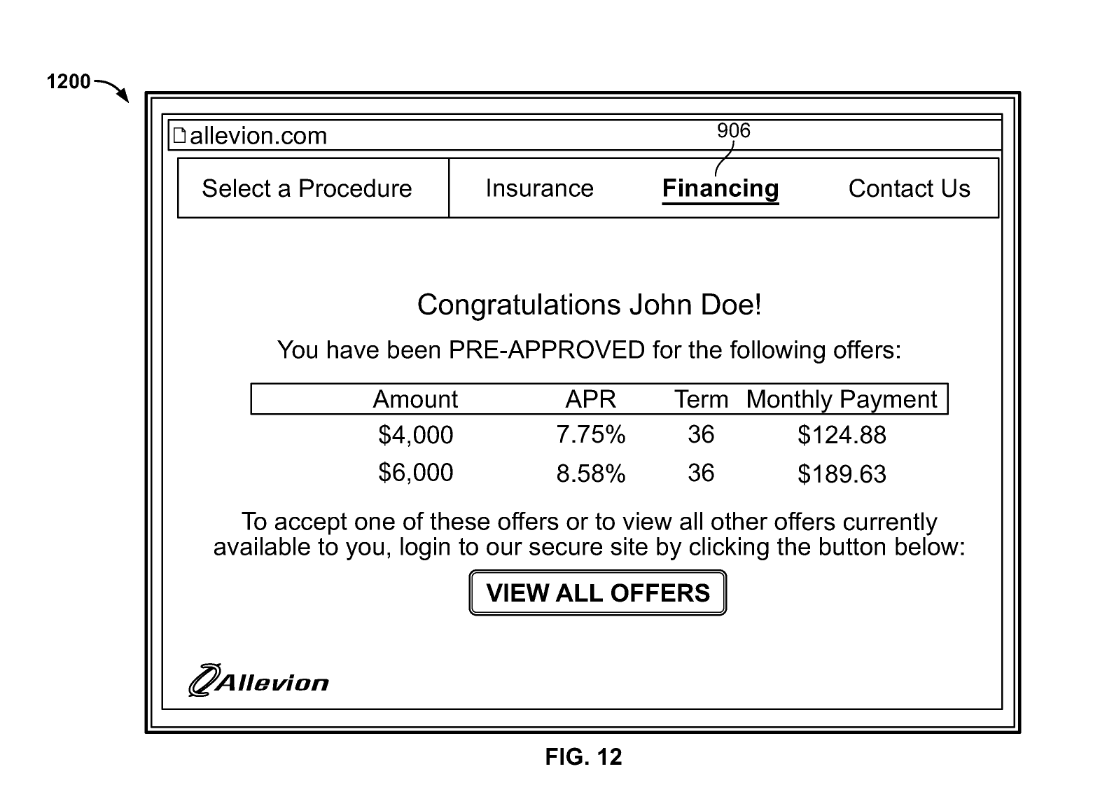

User interface 800 also permits the uninsured user to obtain financing for the selected procedure. FIG. 12 shows an example web page 1200 showing financing offers that have been preapproved for a patient. Web page 1200 shows that two offers have been approved for the patient—one for $4,000 and another for $6,000. Other information is shown for the offers including APR, term of offer and monthly payment.

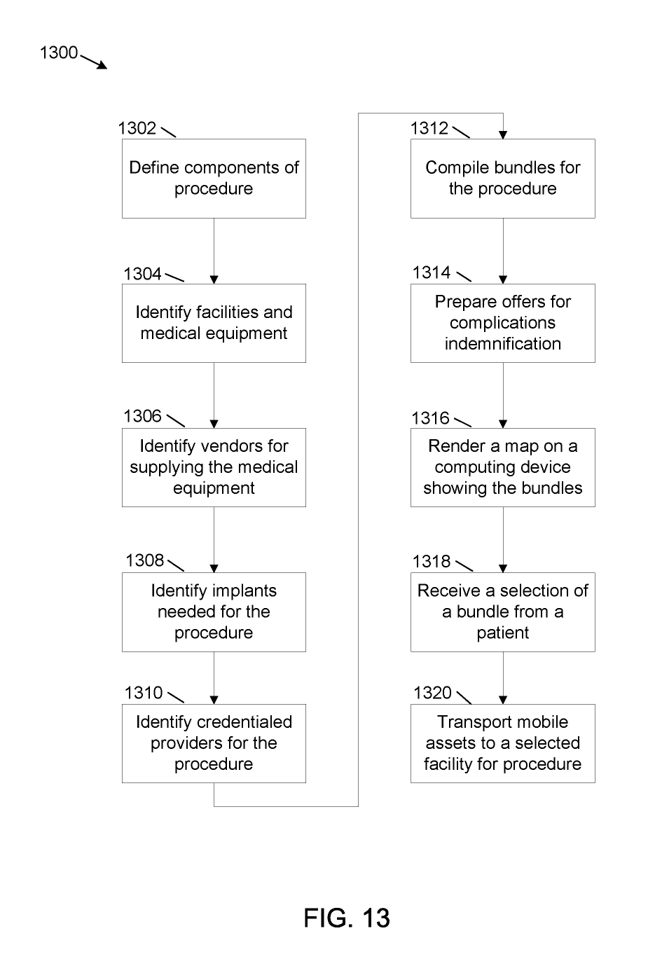

FIG. 13 shows a flowchart of an example method for bundling the components of invasive and/or operative procedures. Examples of procedures that may be effectively composed by bundling their components include total knee replacements, hip replacements, hysterectomies, gastric sleeves and colonoscopies.

At operation 1302, the required components of an invasive and/or operative procedure are defined. These can include, for example, the surgeon, facility, anesthesia, implant, etc.

At operation 1304, the available facilities for invasive and/or operative procedures are identified. The facilities are typically hospitals, ambulatory surgery centers, endoscopy centers, radiology centers, cardiac catheterization laboratories, or provider offices. The identified facilities are ones that have excess capacity and that may be used for bundled invasive and/or operative procedures. The facilities are identified for specific times and dates at which they are available. The facilities may be located in different cities, but are typically located in a defined geographical area such as a state.

In addition, equipment needed for the invasive and/or operative procedures is identified. Some of the equipment may be available at the facilities identified at operation 1302. Other equipment may not be available at the facilities identified at operation 1302 and may need to be transported to the facilities identified from other locations.

As part of this operation, a determination is made regarding whether or not a facility already has certain components or whether the price of the bundle will need to include the cost of providing the component for the procedure. For example, some facilities may have certain medical devices already available for use in an operating room. Others may not and would require that the medical devices be relocated to that operating room for the procedure.

At each of these steps, the prices associated with each component are also evaluated to determine the overall price of the associated bundle. This pricing component, for example, can be used to evaluate the bundle against a price already offered by the medical facility to determine if prices or additive to the overall price.

At operation 1306, vendors are identified for supplying the identified equipment that may not be available at the identified facilities. The vendors selected are ones capable of transporting needed medical equipment to facilities selected for the invasive and/or operative procedure.

At operation 1308, implants needed for the invasive and/or operative procedures are identified. Example implants include artificial knees and penile prostheses.

At operation 1310, qualified providers, such as surgeons, are identified for the invasive and/or operative procedure. Those identified are credentialed and privileged by one or more of the identified facilities. To perform the invasive and/or operative procedure, one or more of the qualified providers can be moved from one facility in one location to another facility at different location. These providers can collectively form a mobile preferred provider organization.

A facility agnostic administrative service enables and accelerates provider mobilization to new facilities; it does this by reducing the time needed to pass new providers through facility medical staff offices, credentialing committees, medical executive committees, board level privileging sub-committees, and other bureaucratic bodies. It does this by collecting commonly-required credentialing and privileging information, such as provider name and address; collecting and verifying primary documents, such as medical school diplomas; and presenting these materials in an organized fashion to identified facility. This service supports facility based credentialing and privileging processes or accepts authority delegated to it for credentialing and privileging by the facility.

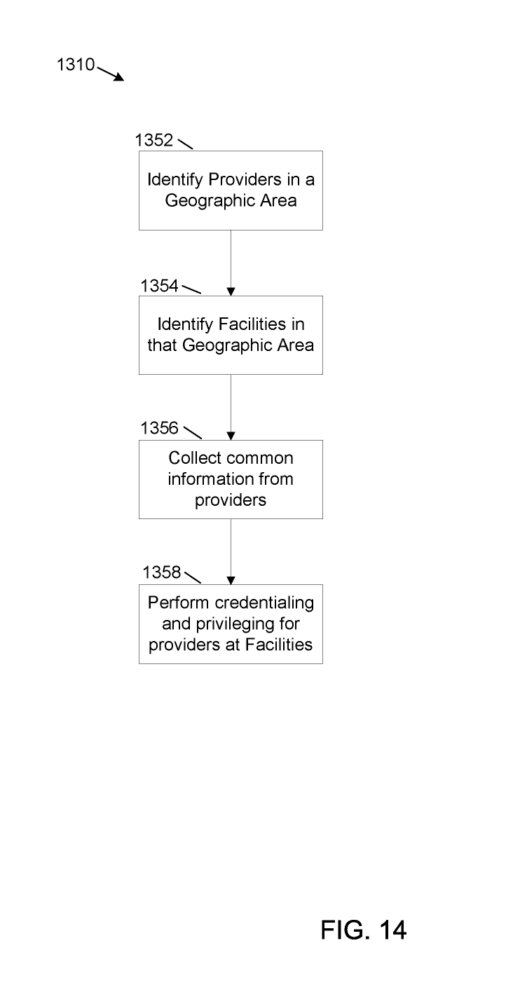

For example, as shown in FIG. 14, in one embodiment, the facility agnostic administrative credentialing and privileging service is presented to and engaged by providers as a virtual clearinghouse, through an online, interactive portal that can accept information, attestations, copies of documents, photographs, and related material for processing by the virtual medical office staff and/or transmission to a new facility for processing. The virtual medical staff office is configured to accelerate the placement of providers in different facilities in different locations. This can include, for example, obtaining credentials and/or privileges for multiple providers at multiple facilities.

Specifically, at operation 1352, the providers in a geographic area are identified. Next, at operation 1354, the facilities in that geographic area are identified. At operation 1356, information is collected from the providers that is needed for the credentialing and privileging. This can be accomplished, as noted, using an online portal or other efficient mechanism. For example, the information can be collected when a provider signs up to participate in the credentialing and privileging program. Finally, at operation 1358, the credentialing and privileging is performed in a batch style at the facilities to streamline the process.

Such a method allows for creation of the bundled and privileged provider along with the medical facility and associated price for an invasive and/or operative procedure on a computing device. The method also allows the provider to be mobile, i.e., travel to new and/or different medical facilities at different locations.

Referring again to FIG. 13, at operation 1312, components of invasive and/or operative procedures are synchronized, coordinated, and matched so as to form complete service bundles. The invasive and/or operative procedure bundles include combinations of facilities, providers, equipment, implants and available dates for the invasive and/or operative procedures. A price is also assigned to each bundle. For an uninsured patient or a payor, such as an insurance company, this is a price that is to be paid to the organization that provides the bundle. For an insured patient, the price is an out-of-pocket cost, based on the terms of his/her insurance policy.

At operation 1314, offers are prepared for complications protection for each of the service bundles. The complications protection protects patients against the economic losses that are associated with complications that may occur during or after the invasive and/or operative procedure.

At operation 1316, an interactive map is rendered on a computing device showing the bundles. Markers are sometimes used to identify locations on the map in which the bundles are available. A user can research a bundle by clicking on a marker to reveal such details as associated price and involved providers. In other embodiments, selected features are displayed directly on the map. For example, the marker can be associated with and/or replaced by the price figure in a way that does not require any more clicking to show the map and money.

At operation 1318, a selection of a bundle is received from a patient. When a user selects an invasive and/or operative procedure bundle and signs up for it, the user may be asked to fill out additional forms, for example health history forms, etc. and may also be directed to set up an appointment to visit the provider that is to perform the procedure. Information solicited will vary based upon the procedure selected. For example, a knee replacement will require different specifics of a patient's medical history than a bariatric procedure. The forms that appear upon selection of a procedure will populate based upon the selection. In addition, surgeons have varying preferences regarding medical records needed to properly “remote-qualify” a patient. For example, with total knee replacement, Physician A may prefer a standing, weight-bearing knee x-ray whereas Physician B prefers an MM.

At operation 1320, when the bundle is selected, along with arrangements to transport the providers, arrangements are made to transport mobile assets, such as medical equipment and implants, to the bundled facility so that the mobile assets arrive at the selected facility in time for the start of the procedure. In example embodiments, the system can automate one or more of these tasks. For example, the system can be programmed to automatically schedule patient visits and schedule logistics associated with the physician, location, and/or surgical components.

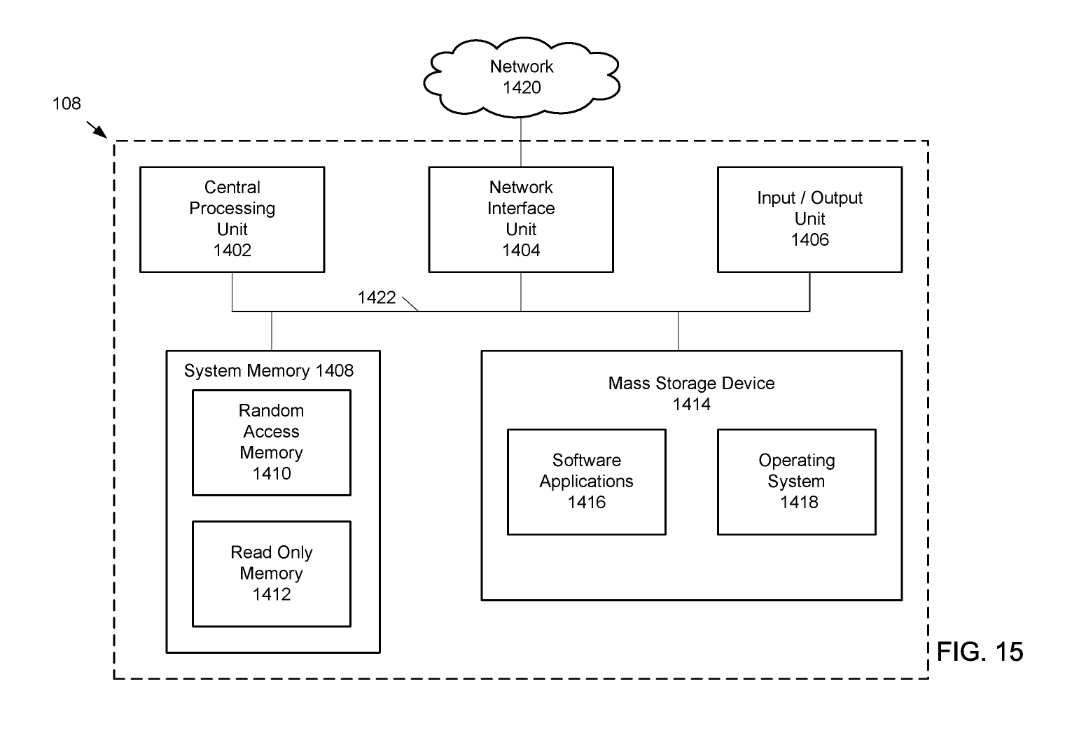

As illustrated in the example of FIG. 15, web server computer 108 includes at least one central processing unit (“CPU”) 1402, a system memory 1408, and a system bus 1422 that couples the system memory 1408 to the CPU 1402. The system memory 1408 includes a random access memory (“RAM”) 1410 and a read-only memory (“ROM”) 1412. A basic input/output system that contains the basic routines that help to transfer information between elements within the web server computer 108, such as during startup, is stored in the ROM 1412. The web server computer 108 further includes a mass storage device 1414. The mass storage device 1414 is able to store software instructions and data. A central processing unit, system memory and mass storage device similar to that in FIG. 15 are also included in client computers 102, 104 and 106.

The mass storage device 1414 is connected to the CPU 1402 through a mass storage controller (not shown) connected to the system bus 1422. The mass storage device 1414 and its associated computer-readable data storage media provide non-volatile, non-transitory storage for the web server computer 108. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the web server computer 108.

According to various embodiments of the invention, the web server computer 108 may operate in a networked environment using logical connections to remote network devices through the network 1420, such as a wireless network, the Internet, or another type of network. The web server computer 108 may connect to the network 1420 through a network interface unit 1404 connected to the system bus 1422. It should be appreciated that the network interface unit 1404 may also be utilized to connect to other types of networks and remote computing systems. The web server computer 108 also includes an input/output controller 1406 for receiving and processing input from a number of other devices, including a touch user interface display screen, or another type of input device. Similarly, the input/output controller 1406 may provide output to a touch user interface display screen or other type of output device.

As mentioned briefly above, the mass storage device 1414 and the RAM 1410 of the web server computer 108 can store software instructions and data. The software instructions include an operating system 1418 suitable for controlling the operation of the web server computer 108. The mass storage device 1414 and/or the RAM 1410 also store software instructions, that when executed by the CPU 1402, cause the web server computer 108 to provide the functionality of the web server computer 108 discussed in this document. For example, the mass storage device 1414 and/or the RAM 1410 can store software instructions that, when executed by the CPU 1402, cause the web server computer 108 to display received financial data on the display screen of the web server computer 108.

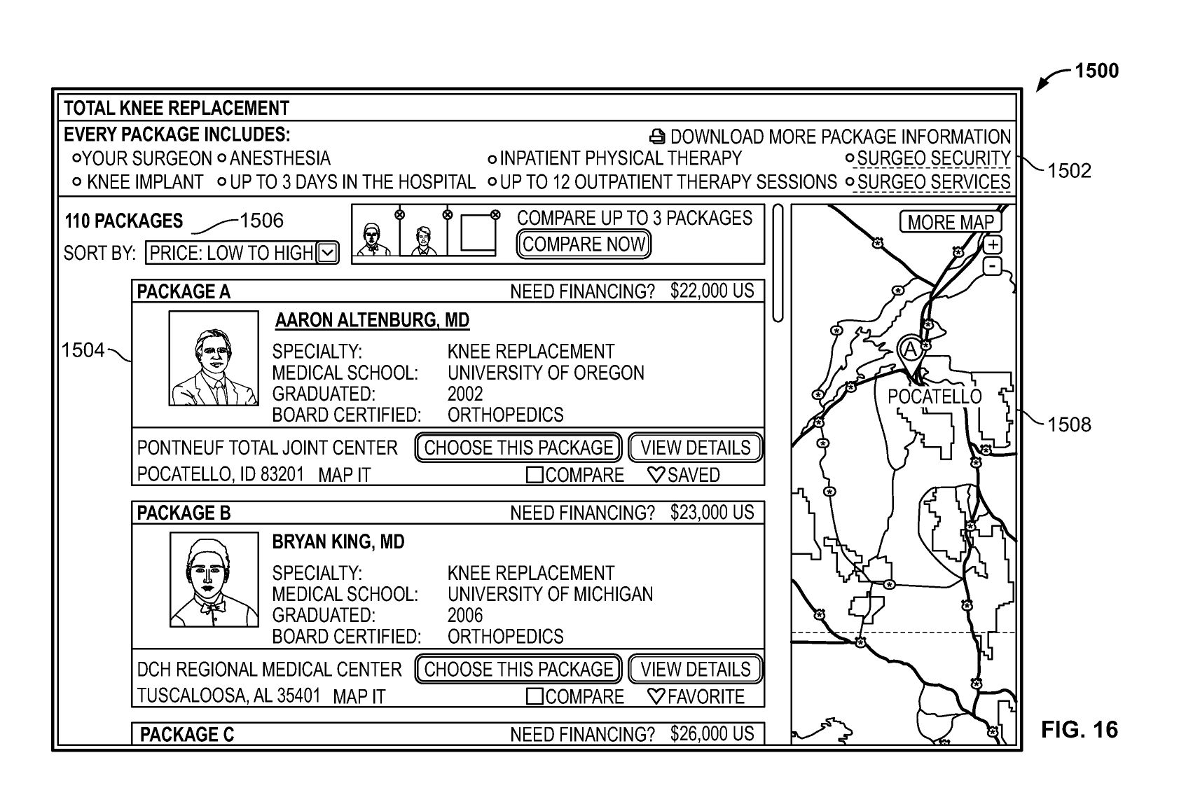

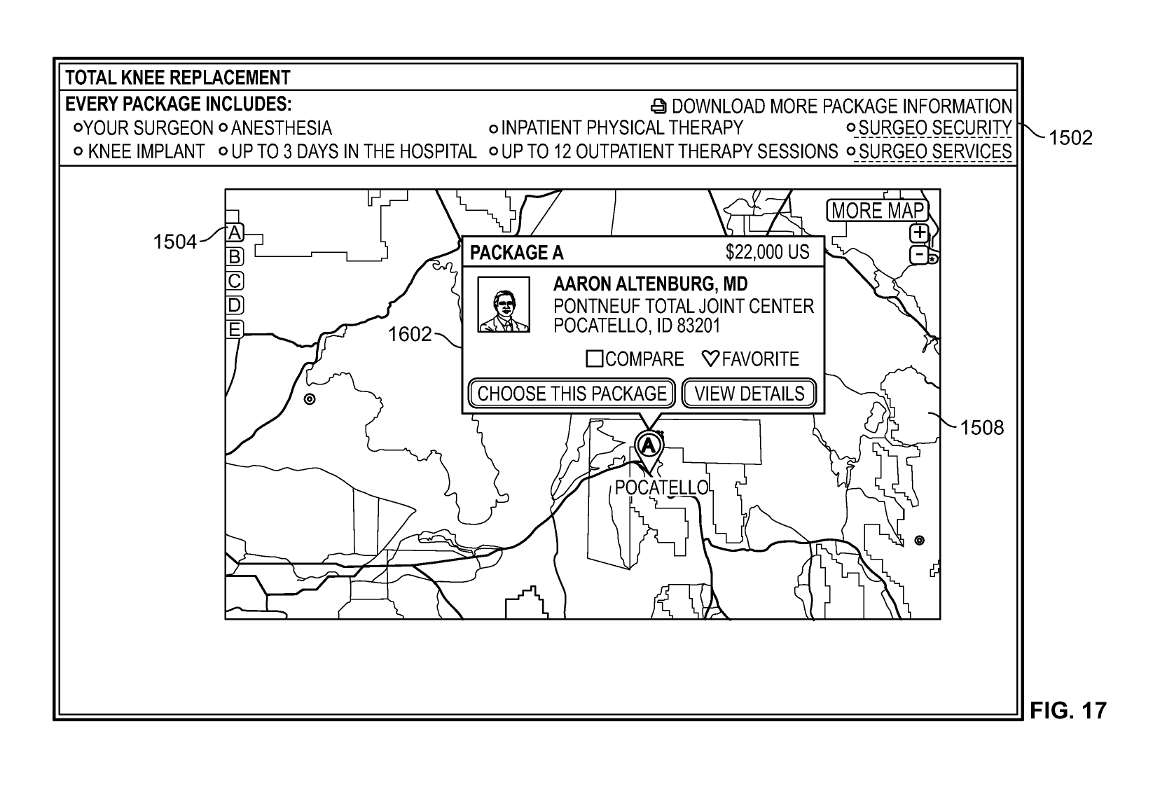

Referring now to FIGS. 16-17, another example of a user interface 1500 is shown that allows an end user to search for bundled invasive and/or operative procedures. The user interface 1500 is similar to that of the interface 300 described above, in that the interface 1500 provides a visual representation of one or more bundled invasive and/or operative procedures within a geographic region.

In this example of FIG. 16, the interface 1500 includes a detailed description 1502 of the aspects associated with the selected bundled invasive and/or operative procedure, along with a list 1504 of the available providers and a map 1508 illustrating the location of the providers. In addition, the interface 1500 provides filters 1506 that allow the bundled invasive and/or operative procedures in the list 1504 to be filtered and sorted in different manners. For example, the list 1504 can be sorted by price, location, etc. And, various provides can be selected and compared.

FIG. 17 shows an enlarged view of the map 1508, with a single provider 1602 highlighted. End users can select between different providers using the abbreviated list 1504.

Alternative embodiments are possible. For example, in another embodiment, the system can be used to accommodate bundled invasive and/or operative procedures associated with a particular provider or group of providers.

For example, a particular institution, such as a medical insurance provider and/or hospital, may desire to provide end users with information about the bundled invasive and/or operative procedures that the end user can select from the particular provider. In such a scenario, the system can be programmed to provide the end user with a branded experience associated with that particular provider. The end user accesses a site specifically-branded for the provider, and the end user is able to search for browse, and select one or more of the bundled invasive and/or operative procedures for that provider. This is accomplished by the system performing the methodology described herein and is transparent to the end user.

In such a “white-label” scenario, the provider can pay for the use of the system. The hosting of the data associated with the provider's bundled invasive and/or operative procedures can be handled and maintained by the system. In some examples, the provider can access the system to provide updates to the data associated with the bundled invasive and/or operative procedures listed within the system. In this example, the provider can be provided with a portal that allows the provider access to update data as well as receive information about the end users who have selected bundled invasive and/or operative procedures from the provider.

In addition to coordinating and pricing bundles for invasive and/or operative procedures, methods and systems are described for analyzing and visually depicting participant behavior within a market for invasive and/or operative procedures. Movements of participants—patients, medical providers, medical equipment, and others—over time and space to and from the relatively, although not absolutely, fixed locations of the facilities that are the site of these invasive and/or operative procedures recorded, compiled, and visually depicted on static and/or dynamic basis to provide insights into their behavior.

Health care administrators, insurance companies, medical providers, facility operators, equipment distributors, government officials, policy analysts, health economists, behavioral economists, psychologists, and other participants in and/or observers of the market can utilize information about the geographic distribution and behavior of facilities and participants within the market to identify opportunities for improvements and optimizations that can be made to the delivery of and/or access to invasive and/or operative procedure bundles for the benefit of the participants. The visual depictions can be static and/or dynamic, and the visual depictions may be customized based upon various facility, patient, provider, equipment, payer, procedure, and other participant criteria. The invasive and/or operative procedures may be part of a bundle or not.

The system depicted in FIG. 1 and described above also supports the visualization of geographic distributions of facilities and participants in a market for invasive and/or operative procedure bundles. Facilities are locations at which invasive and/or operative procedures take place, e.g., an ambulatory surgery center. Participants include patients, medical providers, and medical equipment distributors. The client computers 102, 104, 106 may be utilized by health care administrators, insurance companies, medical providers, facility operators, equipment manufacturers, government officials, policy analysts, health economics, behavioral economists, psychologists, and other participants in the system to analyze the behavior of participants within the market.

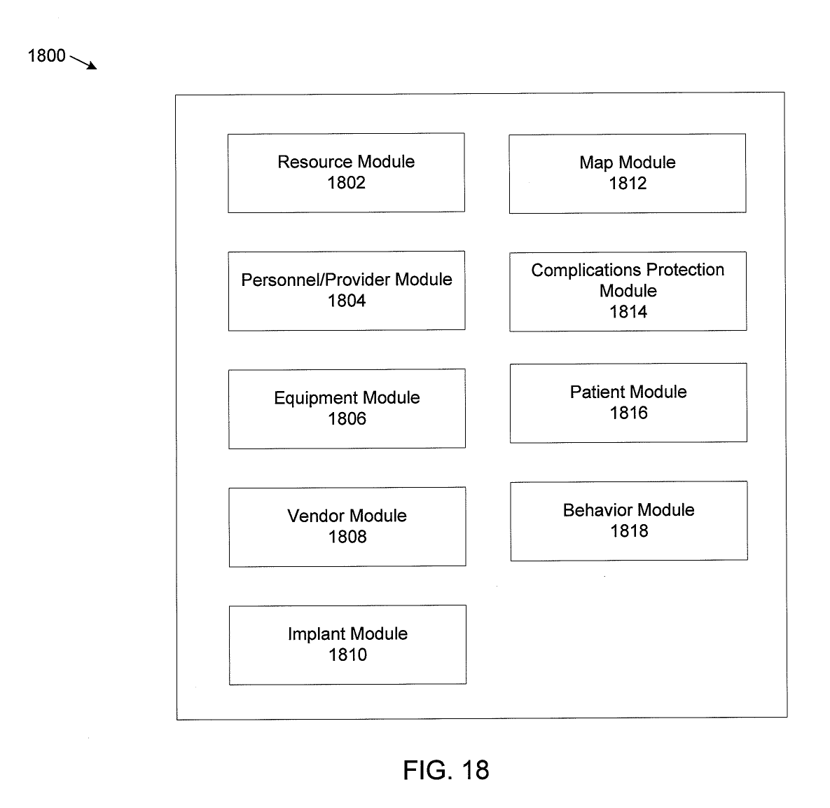

FIG. 18 shows an alternative example of a web server computer 1800 that is programmed and configured in a manner similar to that of the web server computer 108 of FIG. 2. The alternative web server computer 1800 includes a resource module 1802, a personnel module 1804, an equipment module 1806, a vendor module 1808, an implant module 1810, a map module 1812, a complications protection module 1814, as well as a patient module 1816 and a behavior module 1818.

The resource module 1802 in FIG. 18 corresponds to the resource module 202 in FIG. 2 and is programmed to perform the same or similar functions. Additionally, the resource module 1802 can identify the locations of facilities which may be used and/or have been used to perform invasive and/or operative procedures.

The personnel module 1804 corresponds to the personnel module 202 in FIG. 2 and performs the same or similar functions described above. The personnel module 1804 can also identify the geographic location of origin for medical providers and determine whether a provider needs to travel to perform a procedure at a particular facility. The personnel module 1804 may also identify sociodemographic and professional attributes of providers. Such attributes may include gender of the provider, age of the provider, number of years in practice, the provider's type of practice, the provider's medical specialty, ratings for the provider, where the provider attended school, etc.

The equipment module 1806 corresponds to the equipment module 206 and is programmed to perform the same or similar functions. Additionally, the equipment module 1806 identifies economic and brand attributes of medical equipment needed to perform a procedure. These attributes include brand, cost, type of company, ratings, etc.

The vendor module 1808 corresponds to the vendor module 208 of FIG. 2 and performs the same or similar functions.

The implant module 1810 corresponds to the implant module 210 of FIG. 2 and is programmed to perform the same or similar functions. Similar to the equipment module 1806, the implant module 1810 is also programmed to determine economic and brand attributes of implants needed to perform invasive and/or operative procedures.

The map module 1812 corresponds to the map module 202 of FIG. 2 and is programmed to render an interactive map. In addition to showing the locations where procedures are available, the map module 1812 indicates the geographic distribution of patients, providers, facilities, and medical equipment. The map module 1812 interfaces with the resource module 1802, personnel module 1804, equipment module 1806, vendor module 1808, implant module 1810, and patient module 1816 to assemble location information for all participants in an invasive and/or operative procedure bundle market.

The map module 1812 assembles information about facility types in addition to facility locations. Types of facilities may include hospitals, ambulatory surgery centers, physician offices, free-standing emergency rooms, free-standing colonoscopy centers, and the like. The map module 1812 may also visually distinguish between markers on the map representing patients, providers, facilities, and equipment based on their attributes.

The complications protection module 1814 corresponds to the complications protection module 214 of FIG. 2 and performs the same or similar functions.

The patient module 1816 is programmed to identify patients that are participating in the invasive and/or operative procedure market. The patient module 1816 determines a geographic location associated with each patient. Generally, this could be the patient's home address, but could also be a temporary residence or another address associated with a patient, such as the patient's temporary home, place of work, etc. The patient module 1816 is also programmed to characterize patients by sociodemographic and clinical attributes. These attributes may include one or more of the following: gender, age, body mass index, income, method of payment, insurance status, ethnicity, and the like.

The behavior module 1818 is programmed to calculate distances between patients, providers, equipment, and facilities. The behavior module 1818 determines the activity density of procedures completed for a specific package, i.e., the number of procedures completed for that specified invasive and/or operative procedure bundle. The behavior module 1818 can determine the movements and behaviors of participants in the market for the invasive and/or operative procedure. The behavior module 1818 operates in conjunction with the map module 1812 to display visual depictions representing participant movements; static and/or dynamic visual depictions may be constructed when based upon scales measuring calendar time, patient age, and/or other criteria, e.g., time-lapse (video) participant behavior.

For example, if two locations offer a specific invasive and/or operative procedure, the behavior module 1818 is programmed to illustrate the activity density for that procedure. In such an instance, the activity density over, for example, time, can be depicted visually to illustrate how participants in the market are selecting between the two procedures over time. Other attributes, such as patient demographics, practitioner attributes, etc., can similarly be depicted in a static and/or dynamical visualization of activity density, as described further below.

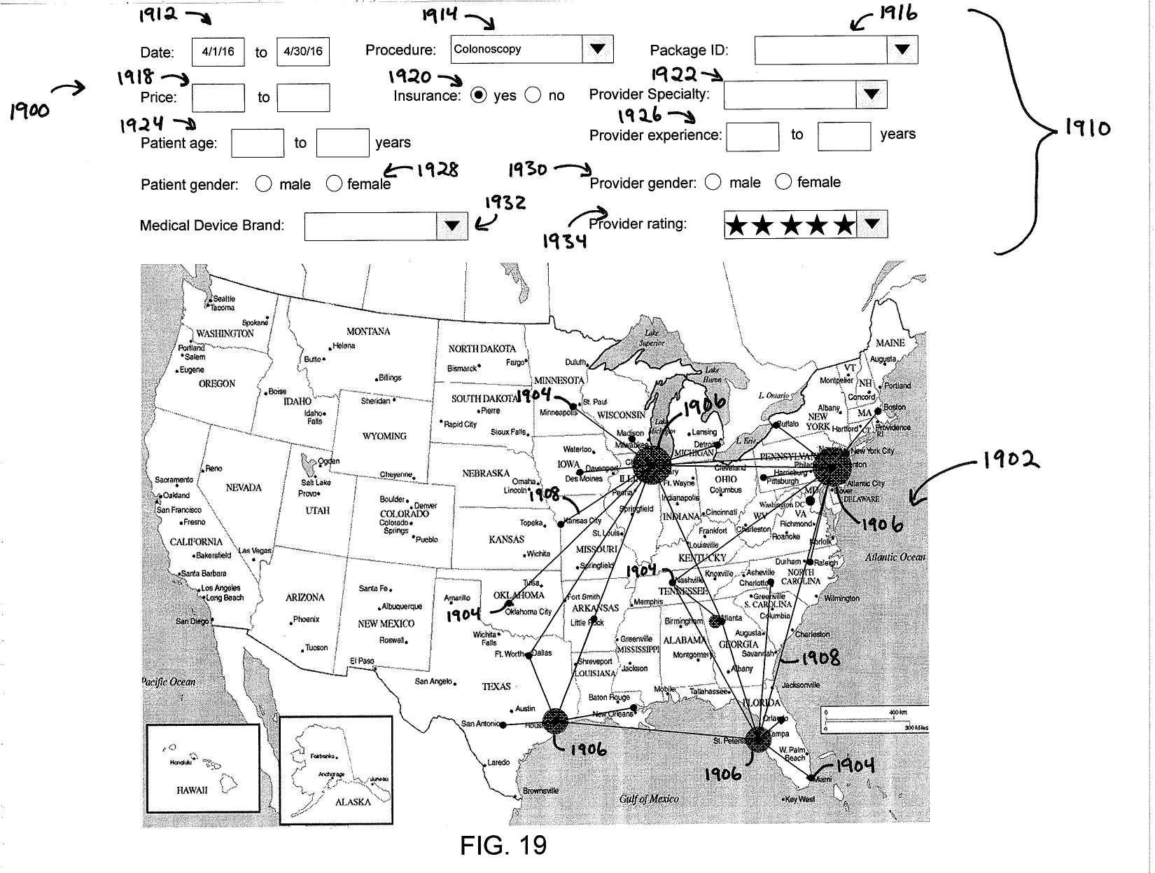

FIG. 19 shows an example user interface 1900 that may be rendered for the end user, by the map module 1812 and the behavior module 1818, on one of the client computers 102, 104, 106, 107 using, for example, a web browser or standalone software application. Exemplary users include hospital administrators, insurance providers, healthcare companies, or medical equipment distributors. The user interface 1900 displays an interactive map 1902 of a geographic area (here, the United States) and a plurality of selectors 1910 for defining the parameters of the display.

The interactive map 1902 displays markers 1904 associated with one of a bundled invasive and/or operative procedure, a patient, a healthcare provider, or equipment. The markers 1904 associated with invasive and/or operative procedures are displayed at a geographical location of a facility at which the procedure is to be performed. Markers 1904 associated with patients, healthcare providers, and equipment are displayed at a geographical location at which the patient/provider/equipment originates. For example, in FIG. 19 there are markers 1904 at Miami, Oklahoma City, Minneapolis, and Nashville indicating the location of a facility, a provider, a patient, or equipment.

Distances between markers are indicated with travel lines 1908. These indicate that a patient, provider, or equipment needs to travel to a facility for an invasive and/or operative procedure. For example, in FIG. 19 there is a travel line 1908 between Kansas City and Chicago indicating movement of a participant in the bundled procedure market. Distances between participants and facilities are calculated and an activity density of bundled procedures is determined.

The activity density of procedures is indicated on the interactive map 1902 by density indicators 1906. The activity density indicators 1906 may be shown with graphical indicators that correspond in size to the number of procedures that occur within that geographical area. For example, in FIG. 19 there are activity density indicators 1906 at Chicago, Philadelphia, Tampa, Atlanta, and Houston. The activity density indicator 1906 at Atlanta has a small diameter and is relatively translucent, signifying that a relatively small number of procedures take place in Atlanta. The activity density indicators 1906 for the Chicago and Philadelphia bundles have larger diameters and are relatively opaque, signifying that a greater number of bundles are consumed by patients, i.e., they are more active.

A user may filter the results displayed on the interactive map 1902 by utilizing the selectors 1910 to narrow the results based on various criteria. Selectors may include drop down menus, text entry fields, check boxes, sliders, and/or radio buttons. For example, in FIG. 19 there is date range field 1912, a procedure selector 1914, a package ID selector 1916, a procedure cost range field 1918, an insurance selector 1920, a provider specialty selector 1922, a provider years of experience range field 1926, a patient gender selector 1928, a provider gender selector 1930, a medical device brand selector 1932, and a provider rating selector 1934. Here, a date range of Apr. 1, 2016 to Apr. 30, 2016 has been entered into the date range field 1912. Colonoscopy has been chosen in the procedure selector 1914. A button indicating that a patient has insurance is selected at the insurance selector 1920. A provider rating of 5 stars has been chosen in the provider rating selector 1934. As a result, only markers 1904, density indicators 1906, and travel lines 1908 corresponding to participants and facilities that meet the criteria chosen in the selectors 1910 are displayed.

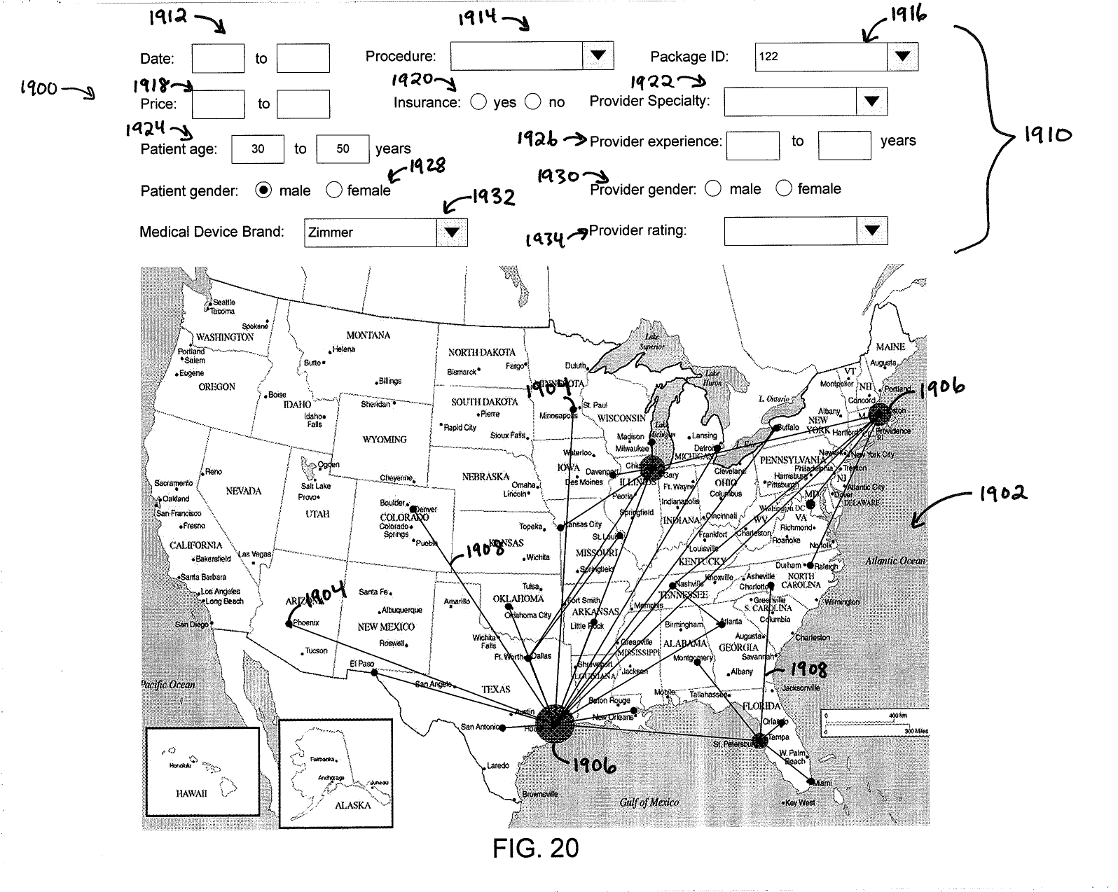

The interactive map 1902 displays participant behavior based on the chosen criteria. For instance, in FIG. 20 different selectors 1910 have been chosen by a user. Here, 122 has been chosen at the package ID selector 1916, a patient age range of 30 to 50 years has been entered at the patient age selector 1924, male has been chosen at the patient gender selector 1928, and Zimmer has been chosen at the medical device brand selector 1932. As a result, a different set of markers 1904 are displayed on the interactive map 1902. For instance, there are now markers 1904 shown at El Paso, Phoenix, and Denver but there are no longer markers 1904 at Pittsburgh or Montgomery. The density indicators 1906 have also shifted based on the different criteria. There is no longer an activity density indicator 1906 at Philadelphia or Atlanta (alternatively, a null indicator may be shown, e.g. empty, fully transparent circle). Instead, a new activity density indicator 1906 now appears at Boston and the density indicator 1906 at Houston has grown in diameter.

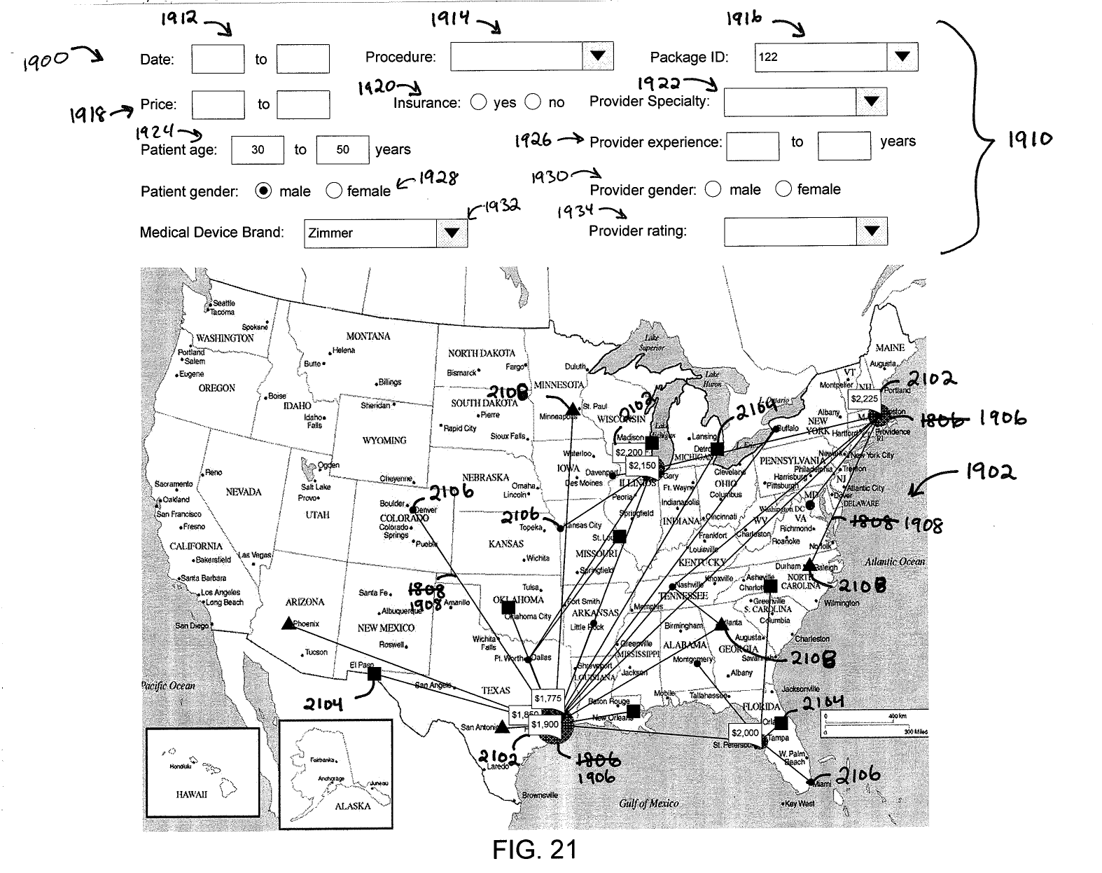

FIG. 21 shows another view of the user interface 1900 with the same selectors 1910 chosen as in FIG. 20. However, in this view the generic markers 1904 are replaced with facility markers 2102, patient markers 2104, provider markers 2106, and equipment markers 2108. The different markers can be distinguished by color, shape, pattern, or picture. In the example shown in FIG. 21, facilities are represented by facility markers 2102 and can also include information like prices for the selected invasive and/or operative procedure bundle(s). The prices shown indicate the total cost to a patient for a particular invasive and/or operative procedure bundle that takes place at that geographic location. The price shown on the map for a particular procedure bundle corresponds to an out-of-pocket cost if for an insured patient or a total bundle price if for an uninsured patient. Prices may also be shown for individual procedures. The facility markers 2102 may also be distinguished by color, shape, pattern, or picture based on the type of facility shown by the marker.

The location of patients is indicated by square patient markers 2104. Medical providers are shown with circular provider markers 2006 indicating their point of origin. Medical equipment including devices and implants are shown at their points of origin with triangular equipment markers 2108. For example, for a procedure bundle having package ID 122 using a Zimmer device, there may be a procedure that takes place with a 45-year-old man in Houston that costs $1,900. This is shown on the interactive map 1902 at a facility marker 2102 located at Houston. Travel lines 1908 indicate that a patient traveled from El Paso, a piece of equipment was obtained from a vendor in Phoenix, and a medical provider traveled from Dallas to Houston to perform the procedure.

Facility markers 2102, patient markers 2104, provider markers 2106, and equipment markers 2108 may be further visually distinguished based their attributes. For example, patient markers 2104 may be visually distinguished by shape or color based on the sociodemographic and clinical attributes of the patients they represent. Similarly, provider markers 2106 may be visually distinguished by shape or color based on the sociodemographic and professional attributes of the providers they represent. Equipment markers 2108 can be visually distinguished with different shaped or colored indicators for economic and brand attributes. Facility markers 2102 may be visually distinguished based on the type of facility.

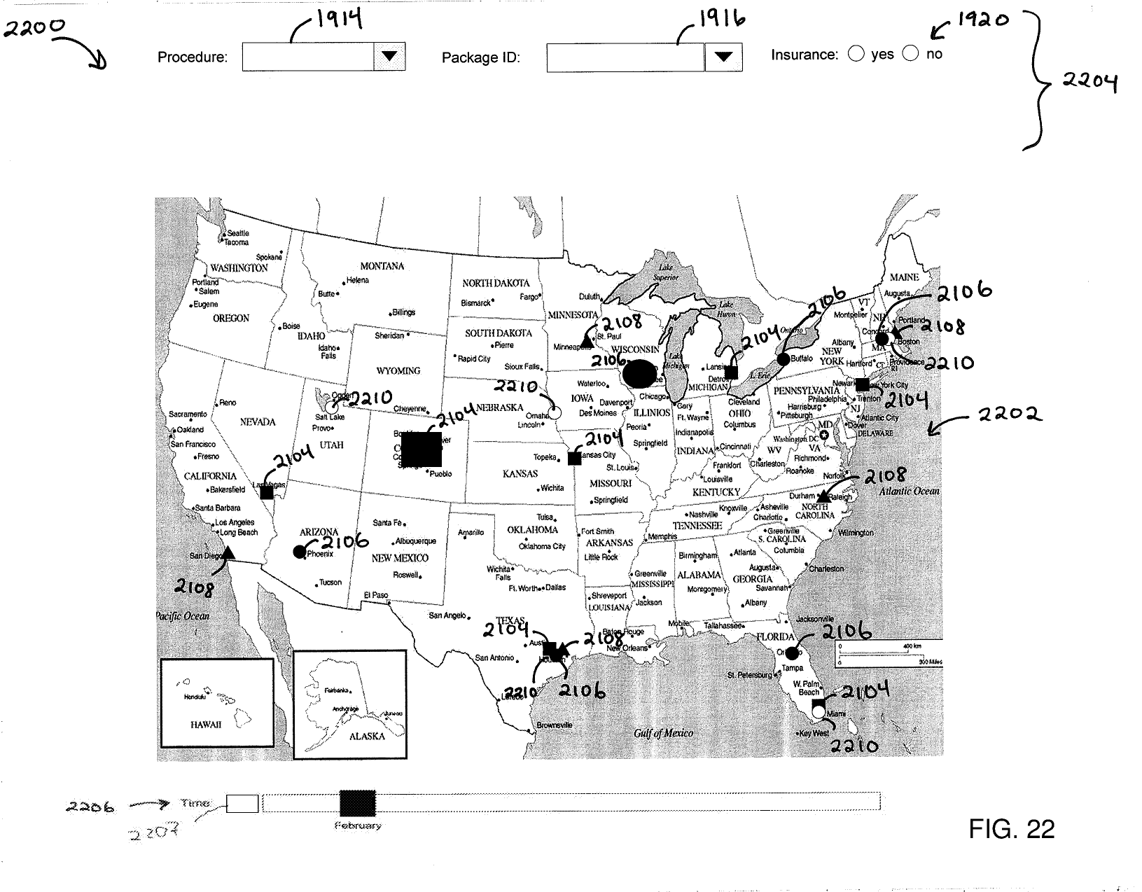

FIG. 22 shows an example of an alternative user interface 2200 which may be used to track the distribution of participants in an invasive and/or operative procedure market over time. An interactive map 2202 is shown depicting a geographical area of interest. Above the interactive map 2202 is a different set of selectors 2204 than the previous example of a user interface 1900. The procedure selector 1914, package ID selector 1916, and insurance selector 1920 may be the same as in FIG. 19. However, the interface 2200 also has a time slider 2206, allowing the interface 2200 to be adjusted as described further below.

The interactive map 2202 displays a plurality of patient markers 2104, provider markers 2106, and equipment markers 2108 similar to FIG. 21. The patient markers 2104 are closed squares, the provider markers 2106 are closed circles, and the equipment markers 2108 are closed triangles. Instead of displaying price tags, the locations of facilities are depicted with facility markers 2210 represented by open circles. The locations of these markers are determined by the time selected on the time slider 2206. Additional limitations may be imposed on which markers are shown by making selections at the procedure selector 1914, package ID selector 1916, and/or insurance selector 1920. In the example shown in FIG. 22, a patient marker 2104, a provider marker 2106, and an equipment marker 2108 are all located with a facility marker 2210 in Houston, indicating that a procedure is taking place there at that time (February).

Other markers are shown by themselves or with only one other marker at one place, indicating the position of the participant in the geographic area when the participant is not involved in a procedure. For example, an equipment marker 2108 is shown by itself in Raleigh indicating its location of origin. A provider marker 2106 is shown by itself in Orlando, indicating the provider's home location. Both a patient marker 2104 and a facility marker 2210 are shown at Miami, indicating that a patient lives in the same place where a facility exists, though the patient may not be in Miami for a procedure that is to take place there.

The activity density for one or more of the items on the interface user interface 2200 is also indicated. For example, the provider marker 2106 shown in Wisconsin is larger than that of other geographies because the number of procedures consumed at that location is larger than at other locations. Similarly, the size of the procedure marker 2104 in Colorado indicates a greater activity density for procedures in that location as compared to others on the interactive map 2202.

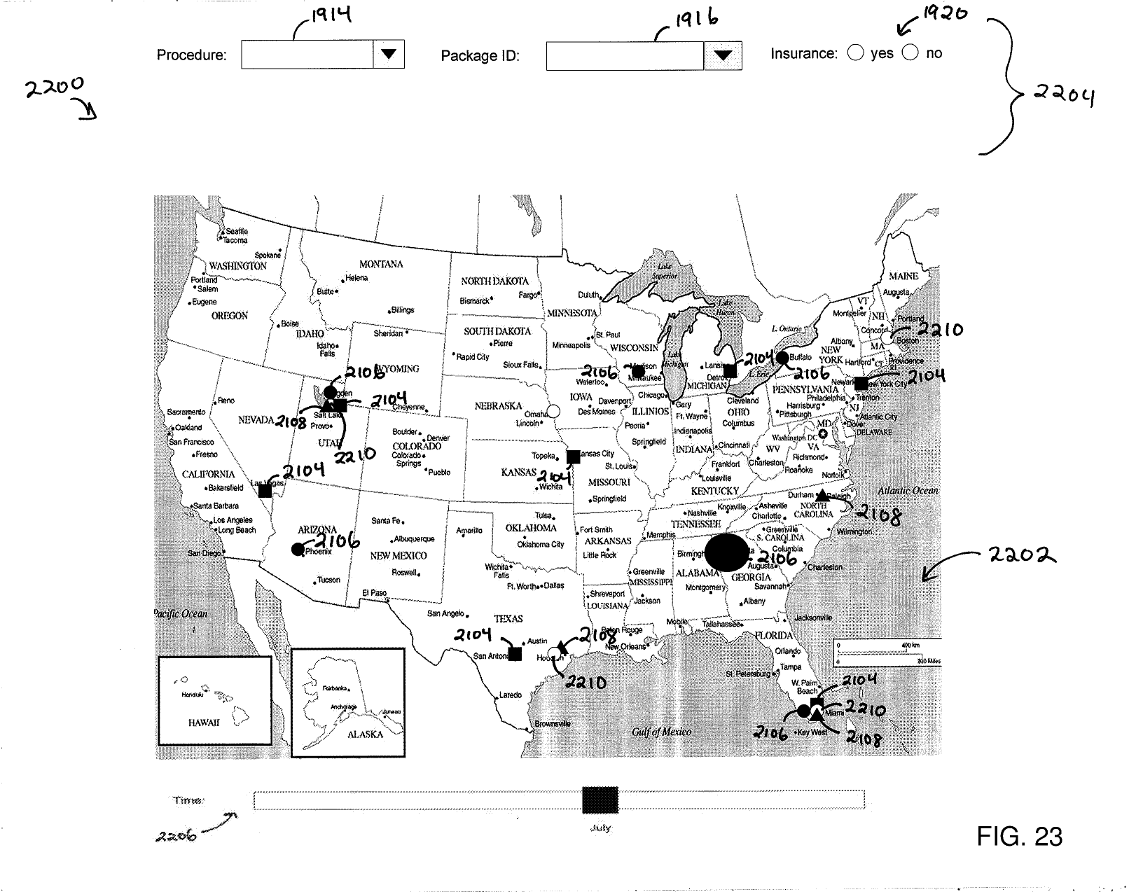

FIG. 23 shows the interactive map 2202 after the time slider 2206 has been adjusted. With adjustments of the time slider, the locations of the markers change to reflect the positions of participants in the invasive and/or operative procedure market at various time points. Here the time slider 2206 has been adjusted to July. Now there is only an equipment marker 2108 and a facility marker 2210 shown in Houston. A patient marker 2104 now appears in San Antonio, indicating that a patient in San Antonio has entered the market. A provider marker 2106 is shown in Atlanta instead of Houston, indicating that a provider has entered the market in Atlanta.

A patient marker 2104, a provider marker 2106, and an equipment marker 2108 now appear in Salt Lake with a facility marker 2210. In FIG. 22 there was only a facility marker 2210 present. This indicates that a procedure is taking place in Salt Lake for a patient from Denver with equipment from San Diego, as the markers that were previously in Denver and San Diego in FIG. 22 are now in Salt Lake in FIG. 23.

Further, the activity density magnitudes associated with procedure bundles are depicted. For example, the procedure marker 2106 shown in Wisconsin has shrunk because the number of procedures consumed at that location is less than at the time shown in FIG. 21. By a similar token, the activity density for procedures in Georgia has a greater significance.

By adjusting selectors 2204, a user can visualize movements of the mobile participants in an invasive and/or operative procedure market in comparison to the relatively fixed geographic locations of facilities at which procedures are performed. In other words, depending on the variable selected, the depictions of the activity densities for those variables are modified.

The time slider 2206 can also show other measurements of time, such as the age of patients, in order to determine trends over the average lifespan of a patient. The dynamic visualizations of information about the movement of patients, providers, and equipment can provide insight into the behaviors of participants within the market over time.

In some examples, the time slider 2206 can be adjusted manually, as described above, to show a map at a particular point in time. In others, the time slider 2206 can be automated, so that time elapses and the indications are the map are animated as the time slider 2206 moves along from a first time to a second time. With automation, serial static visual depictions are transformed to a seemingly continuous or semi-continuous dynamic visual depiction. For example, the user can select a start button 2207 to start the dynamic depiction, and then the map can be displayed as a time-lapse video with the depictions changing over time as the time slider 2206 automatically moves from the first time to the second time. This dynamic depiction of the map over time gives the user an understanding of time-dependent participant behaviors that may not be easily apparent in serial static depictions. This can allow new insights regarding opportunities for system improvement and optimization.

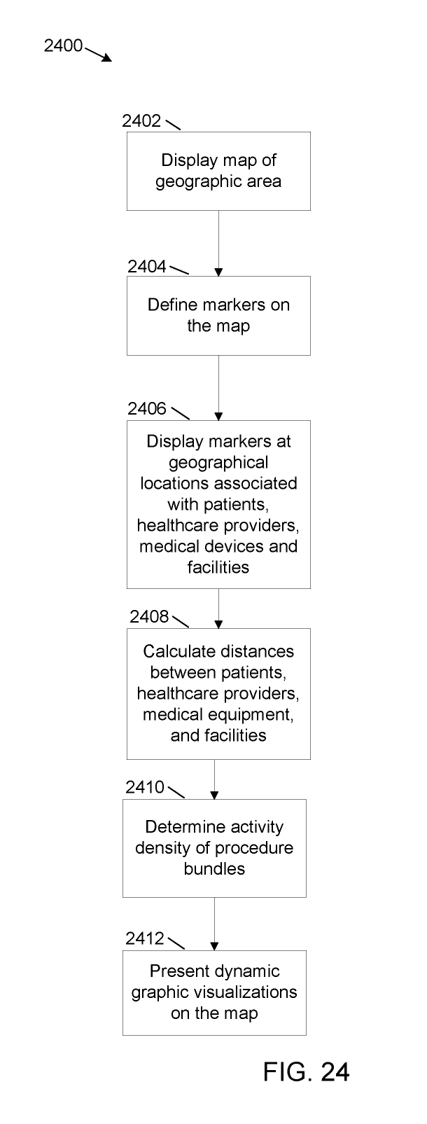

FIG. 24 shows a flowchart 2400 of an example method for visualizing a geographic distribution of participants and/or facilities in a market for invasive and/or operative procedures. A dynamic visual representation of the locations and movements of patients, medical providers, and medical equipment is displayed for a user to determine patterns in participant behavior.

At operation 2402, a map of a geographic area of interest is displayed on an electronic computing device. The map may be displayed on a user interface of a website.

At operation 2404, a plurality of markers is defined on the map. Each marker is associated with one of a bundled invasive and/or operative procedure, a patient, a provider, or a piece of medical equipment. The markers for bundled invasive and/or operative procedures may indicate a price associated with each bundle. Exemplary procedures include total knee replacements, hip replacements, hysterectomies, gastric sleeves, and colonoscopies. Other invasive and/or operative procedures are possible. Patients include any person receiving medical treatment in the form of an invasive and/or operative procedure bundle. Providers include surgeons, nurses, anesthesiologists, and other healthcare professionals. Equipment includes medical devices, implants, and other medical implements required to complete an invasive and/or operative procedure.

At operation 2406, markers are displayed on the map at geographical locations associated with the corresponding patient, healthcare provider, medical equipment, or facility. Markers for invasive and/or operative procedures are displayed at a geographical location of a facility at which the procedure is to be performed. Examples of facilities include hospitals, ambulatory surgery centers, endoscopy centers, radiology centers, cardiac catheterization laboratories, clinics, and private provider offices. Facility markers may include a price calculated for an invasive and/or operative procedure. Markers associated with patients are displayed at geographical locations at which the patients originate. Generally, this would be a patient's home address.

Markers associated with providers are displayed at geographical location at which the providers originate. Such locations may include a provider's home address, work address, or other location specified by the provider. Each provider may be associated with more than one originating geographical location. In such instances, the location at which a marker is displayed for a provider may be determined by the provider's schedule, proximity to a location at which a procedure is to be performed, or by preference of the provider. Markers associated with equipment are displayed at geographical locations at which the equipment originates. Medical devices and implants may originate at locations where they are manufactured, at locations where vendors are headquartered, or at medical supply stores. Specialized medical equipment such as lasers, knee implants, and shock wave generators may be transported to the surgical facility from facilities such as warehouses, distribution hubs, hospitals, research centers, universities, etc.

At operation 2208, distances between patients, healthcare providers, equipment, and facilities are calculated. In order for an invasive and/or operative procedure to take place, one or more participants in the market may need to travel to a facility. The electronic computing device functions to determine the distance that each patient, provider, and/or piece of medical equipment needs to travel to arrive at a facility where an invasive and/or operative procedure is to take place. Additionally, the distances between the locations of facilities at which operative procedures are taking place are calculated.

At operation 2410, the number of procedure bundles served is utilized to determine the activity density. The greater the number, the greater the activity density, i.e. the more procedure bundles are consumed.

At operation 2412, dynamic visual depictions are displayed on the map. The visual depictions represent the movements of patients, healthcare providers, and/or equipment relative to the facilities at which invasive and/or operative procedures take place. Transportation of a patient, provider, or equipment may be visualized by a line extending between the originating location of the patient, provider, or equipment and the facility at which the pertinent invasive and/or operative procedure is taking place. Activity density of procedures may be represented by density indicators, such as circles. The activity density indicators can indicate the level of density at a particular location by the intensity of shading or diameter of a circular indicator.

A user may select to display procedure bundles based on patient attributes (gender), provider attributes (gender), equipment attributes (competitors), facility attributes (hospital vs. surgery center), procedure attributes (open vs. laparoscopic), or time ranges (Mondays vs. Fridays). For example, a user may wish to compare the movements of participants in an invasive and/or operative procedure market for procedures involving a female patient versus a male patient. By changing the criteria from female to male on the user interface, the visualizations on the map may change, indicating different behaviors of patients, providers, and equipment distributors relative to facility locations. In another example, a user may wish to track the movements of all participants over time. By adjusting the time range of the procedures displayed, the user can observe differences in behavior over time. In dynamic embodiments, the movements of participants in the procedure market may be displayed as an automated, time dependent visual depiction, e.g., video, that shows markers changing geographic positions over time and participants behaving differently over time.

Although the example described in conjunction with FIGS. 18-24 is directed to the delivery of invasive and/or operative procedures, the visualization techniques described therein can be used more generally in other markets. For example, the visualization techniques can be used to depict procedures that are bundled in addition to individual invasive and/or operative procedures. For example, the visualization techniques can be used to depict the various attributes (e.g., provider, insurer, patient, etc.) associated with the delivery of medical services in a traditional insured, fee-for-service, i.e. unbundled, environment. Further, the attributes can be applicable outside of the medical field.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A method implemented on an electronic computing device for visually depicting a geographic distribution of facilities or participants in a market for invasive or operative procedures comprising:
on the electronic computing device, displaying a map of a geographic area;
defining a plurality of markers on the map, each marker being associated with an invasive or operative procedure, a facility, a patient, a healthcare provider, equipment, or other participant;
displaying each marker associated with a bundled invasive or operative procedure at a geographical location of a facility at which the procedure is to be performed and displaying each marker associated with a patient, healthcare provider, equipment, or other participant at a geographical location at which the patient, healthcare provider, equipment or other participant originates;
calculating one or more distances between the patient, healthcare provider, equipment, or other participant and the facility at which the procedure is performed;
determining an activity density of procedures performed at each facility; and
presenting visual depictions on the map representing behavior of the patients, healthcare providers, equipment or other participants relative to geographical locations of facilities at which the procedure is performed.

2. The method of claim 1, wherein the markers comprise different colors or shapes to distinguish between types of invasive or operative procedures, patients, healthcare providers, facilities, equipment, or other participant.

3. The method of claim 1, further comprising:
characterizing the patients by sociodemographic and clinical attributes;
wherein the sociodemographic and clinical attributes include one or more of the following: gender, age, body mass index, income, and method of payment; and
wherein patient markers are further distinguished by displaying different shaped or different colored markers corresponding with the sociodemographic and clinical attributes.

4. The method of claim 1, further comprising:
characterizing the providers by sociodemographic and professional attributes;
wherein the sociodemographic and professional attributes include one or more of the following: gender, age, years in practice, type of practice, specialty, and ratings; and
wherein the provider markers are further distinguished by displaying different shaped or different colored markers corresponding with the sociodemographic and professional attributes.

5. The method of claim 1, further comprising characterizing equipment by economic and brand attributes, wherein the economic and brand attributes include one or more of the following: brand, cost, type of company, ratings, and warranty.

6. The method of claim 1, further comprising:
characterizing facilities by type of facility, wherein the type of facility includes one or more of the following: hospital, ambulatory surgery center, physician office, free-standing emergency room, and free-standing colonoscopy center;
wherein facility markers are further distinguished by displaying different shaped or different colored markers corresponding with the type of facility.

7. The method of claim 1, further comprising calculating and showing a price for each procedure bundle on the map, wherein a price shown on the map for the particular procedure bundle corresponds to an out-of-pocket cost if to an insured patient or a total bundle price when to an uninsured patient.

8. The method of claim 1, further comprising:
displaying one or more selectors corresponding to one or more attributes associated with the facility, patient, provider, equipment, procedure bundle, and market as a whole;
wherein the selectors are drop down menus, text entry fields, check boxes, sliders, or radio buttons; and
wherein the attributes include sociodemographic and clinical attributes of patients; sociodemographic and professional attributes of providers; economic and brand attributes of equipment; price and location attributes of procedure bundles; facility types, and calendar time.

9. The method of claim 8, further comprising receiving one or more selections of selectors and updating the visual depictions on the map.

10. The method of claim 9, further comprising:
automatically updating the visual depictions on the map as a function of the one or more selections of selectors;
wherein one of the one or more selections of selectors is time; and
wherein the selections of selectors include manually adjusting a time selector and updating the visual depiction to correspond with movements of patients, providers, and equipment at a different time and provide static visual depictions of behavior of participants over time.

11. The method of claim 10, wherein the selections of selectors include automatic adjustments of the time selector and automatically updating the visual depiction to correspond with movements of patients, providers, and equipment over time and provide a dynamic visual depiction of the behavior of participants over time.

12. The method of claim 10, wherein one of the one or more selections of selectors is patient age.

13. The method of claim 10, wherein the selections of selectors include manually adjusting an age selector and updating the visual depiction to correspond with movements of patients, providers, and equipment at the different time and provide static visual depictions of the behavior of participants with patient age.

14. The method of claim 13, wherein the selections of selectors include automatic adjustments of the age selector and automatically updating the visual depiction to correspond with movements of patients, providers, and equipment over time and provide a dynamic visual depiction of the behavior of participants with patient age.

15. The method of claim 1, wherein the invasive or operative procedure is part of a bundle.

16. A method implemented on an electronic computing device for visually depicting a geographic distribution of facilities or participants in a market for invasive or operative procedures comprising:
on the electronic computing device, displaying a map of a geographic area;
defining a plurality of markers on the map, each marker being associated with an invasive or operative procedure, a facility, a patient, a healthcare provider, equipment, or other participant, and wherein the markers comprise different colors or shapes to distinguish between types of invasive or operative procedures, patients, healthcare providers, facilities, equipment, or other participant;
displaying each marker associated with a bundled invasive or operative procedure at a geographical location of a facility at which the procedure is to be performed and displaying each marker associated with a patient, healthcare provider, equipment, or other participant at a geographical location at which the patient, healthcare provider, equipment or other participant originates;
calculating one or more distances between the patient, healthcare provider, equipment, or other participant and the facility at which the procedure is performed;
determining an activity density of procedures performed at each facility;
presenting visual depictions on the map representing behavior of the patients, healthcare providers, equipment or other participants relative to geographical locations of facilities at which the procedure is performed;
characterizing the patients by sociodemographic and clinical attributes, wherein the sociodemographic and clinical attributes include one or more of the following: gender, age, body mass index, income, and method of payment, and wherein patient markers are further distinguished by displaying different shaped or different colored markers corresponding with the sociodemographic and clinical attributes; and
calculating and showing a price for each procedure bundle on the map, wherein a price shown on the map for the particular procedure bundle corresponds to an out-of-pocket cost when to an insured patient or a total bundle price when to an uninsured patient.

17. A system programmed to present a geographic distribution of facilities or participants in a market for invasive or operative procedures, comprising:
a processing unit; and
system memory, the system memory including instructions that, when executed by the processing unit, create:
a resource module programmed to identify facilities including privileged providers, operating room capabilities, associated procedure fees, facility locations, and facility assets;
a personnel module programmed to identify medical providers qualified, credentialed, and privileged to perform the invasive or operative procedures and determine their originating location;
an equipment module programmed to identify and locate equipment needed for the invasive or operative procedures;
map module programmed to render an interactive map showing locations of available invasive or operative procedure bundles, patient locations, provider locations, facility locations, and medical equipment locations; and
a behavior module programmed to determine an activity density of procedures consumed and present dynamic graphic depictions on the interactive map representing movements of patients, healthcare providers, or equipment relative to facility locations.

18. The system of claim 17, further comprising a vendor module programmed to identify and locate vendors capable of providing some or all of the identified equipment and an implant module programmed to identify and locate implants needed for the invasive or operative procedures.

19. The system of claim 18, further comprising a complications protection module programmed to generate policies to indemnify complications of invasive or operative procedures.

20. The system of claim 19, wherein the dynamic graphic depictions show movements of patients, healthcare providers, or equipment over time.
